SEQUENCE LISTING (2 sequences)

SEQ ID NO: 1 - protein, 498 aa, synthetic construct
  REGION 1..498: note = Description of sequence: BCMA CAR
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 498 aa, synthetic construct
  REGION 1..498: note = Description of sequence: BCMA CAR
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0407252 A1
(43) Pub. Date: Dec. 21, 2023

(54) METHODS FOR DONOR CELL ANALYSIS

(71) Applicant: Allogene Therapeutics, Inc., South San Francisco, CA (US)

(72) Inventors: Meng-Yin LIN, Redwood City, CA (US); Hayung YOON, San Francisco, CA (US); Thomas Charles PERTEL, San Mateo, CA (US); Barbra Johnson SASU, San Francisco, CA (US)

(21) Appl. No.: 18/308,797

(22) Filed: Apr. 28, 2023

Related U.S. Application Data
(60) Provisional application No. 63/486,776, filed on Feb. 24, 2023.
(60) Provisional application No. 63/336,068, filed on Apr. 28, 2022.

(51) Int. Cl. C12N 5/0783; C07K 14/725

(52) U.S. Cl. CPC C12N 5/0636; C07K 14/7051; C12N 2510/00; C07K 2319/03; C07K 2319/02

(57) ABSTRACT

Provided herein are methods, kits and reagents for analyzing the attributes of cell populations, such as donor cells prior to modification to provide engineered cells, e.g., engineered immune cells, such as CAR T cells. For example, provided herein are methods of determining the amount or percentage of biomarkers and/or secretion profiles of donor cell populations, selecting donor cells with certain biomarkers and/or secretion profiles, and engineering the CAR T cells from the selected donor cells.

### CROSS REFERENCE TO RELATED APPLICATIONS

The present application claims the benefit of priority to U.S. Provisional Application No. 63/336,068, filed on Apr. 28, 2022; and U.S. Provisional Application No. 63/486,776, filed on Feb. 24, 2023, the contents of both of which are hereby incorporated by reference in their entireties.

### REFERENCE TO SEQUENCE LISTING

This application contains a Sequence Listing which has been submitted electronically in XML format and is hereby incorporated by reference in its entirety. Said XML copy, created on Apr. 21, 2023, is named AT-052-03US_ST26.xml and is 4,366 bytes in size.

### BACKGROUND

Chimeric antigen receptor (CAR) T cell therapy has achieved unprecedented success, yet manufacturing of CAR T cells also presents unprecedented challenges. CAR T cells derived from a patient's own cells (autologous CAR T cells) have disadvantages including delays in treating patients and the inability to treat all patients due to manufacturing failures stemming from dysfunctional T cells present in this patient population. In contrast, CAR T cells derived from allogeneic donor cells (allogeneic CAR T cells) can be produced as off-the-shelf products with reduced costs and simplified manufacturing process as compared to autologous CAR T cells. Allogeneic CAR T cell therapy uses T cells from healthy individuals as starting material, simplifying supply, and providing off-the-shelf product convenience. Using healthy donors T cells also opens the possibility to optimize therapeutic efficacy by using donor T cells that are immunologically fit and provide a more homogeneous product. There is a need for methods and reagents to identify and select donor T cell populations for use in manufacturing CAR T cell products.

### TECHNICAL FIELD

The instant disclosure relates to methods and reagents for analyzing the attributes of cell populations (e.g., donor cell populations, such as donor immune cell populations and/or engineered cell populations, such as engineered immune cells, e.g., CAR-T cells), selecting suitable donor cell populations for modification to provide engineered donor cell populations, e.g., engineered immune cell populations, such as CAR T cell populations. For example, the instant disclosure relates to, inter alia, methods, compositions, and kits for detecting the presence or absence of biomarkers and/or secretion profiles of donor cell populations, selecting donor cells with certain biomarkers and/or secretion profiles, and engineering CAR T cells from the selected donor cells.

### SUMMARY

The instant disclosure relates to methods and reagents for analyzing the attributes of donor cell populations prior to modification to manufacture engineered cells, e.g., engineered immune cell populations, such as CAR T cell populations. Engineered immune cell populations derived from such donor cell populations may also be analyzed for one or more of the same attributes at one or more timepoints along the engineering process, e.g., the CAR-T cell manufacturing process.

In one aspect, the present disclosure provides methods of manufacturing engineered immune cells. In one embodiment, the method comprises detecting an HLA-DR expression level of 65% or less in an immune cell population. In another embodiment, the method further comprises modifying the immune cell population to express an exogenous nucleic acid sequence, thereby providing an engineered immune cell population. In a further embodiment, the modifying step further comprises reducing or eliminating expression of an endogenous gene (such as for example TCRα and/or CD52 as further described herein).

In other embodiments, (i) the exogenous nucleic acid sequence comprises a chimeric antigen receptor (CAR) nucleic acid sequence or (ii) the exogenous nucleic acid sequence further comprises one or more nucleic acid sequences selected from the group consisting of a chimeric antigen receptor (CAR), a transmembrane domain nucleic acid sequence, a costimulatory domain nucleic acid sequence and a signaling domain nucleic acid sequence. In another embodiment, the exogenous nucleic acid sequence is expressed as a single transcript. In a further embodiment, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one embodiment, the engineered immune cell population comprises, exhibits, shows, or has improved in vitro functionality as compared to a non-engineered immune cell population. In another embodiment, the engineered immune cell population comprises, exhibits, shows, or has improved in vitro functionality as compared to an additional engineered immune cell population that originated from (or was originated from or originated from) an additional immune cell population expressing HLA-DR at a level greater than about 65%. In other embodiments, the improved in vitro functionality comprises one or more of improved in vitro cytotoxicity, improved cell fitness, and reduced cytokine secretion. In one additional embodiment, cytotoxicity is demonstrated by an in vitro killing assay. In other embodiments, the in vitro killing assay comprises the killing of cells that express a target of the CAR. The in vitro killing assay described herein may be a long-term killing assay or a short-term killing assay.

In a further embodiment, the immune cell population is obtained from or derived from a donor prior to the detecting step. In one embodiment, the donor is a healthy donor or a patient in need of treatment (such as for example a human patient). In another embodiment, the patient is a patient in need of treatment with an autologous cell therapy. In one other embodiment, the autologous cell therapy comprises the engineered immune cell population.

In other embodiments, the detecting step comprises detecting a protein level of a molecule, e.g., HLA-DR or TIGIT, using flow cytometry (FACS), an Enzyme-Linked Immunosorbent Assay (ELISA), an immunoblotting assay, an immunofluorescence assay, or an immunochemistry (IHC) assay.

In one embodiment, the donor from which the immune cell population is obtained or derived from prior to the detecting step is a healthy human donor. In another embodiment, the healthy human donor is aged between about 18 and about 30 years old.

In other embodiments, the method further comprises detecting a level of expression of one or more biomarkers selected from the group consisting of TIGIT, CD16, CD56, CCR7, CD27, and CD45RA. In one embodiment, the method further comprises detecting a level of expression of TIGIT. In another embodiment, the TIGIT expression level that is detected is 30% or less in the immune cell population.

In an additional embodiment, the method further comprises depleting HLA-DR-positive immune cells from the immune cell population to provide an HLA-DR-depleted immune cell population and/or depleting TIGIT-positive immune cells from the immune cell population to provide a TIGIT-depleted immune cell population. In one embodiment, the depleting step is performed prior to the modifying step.

In one other aspect, the present disclosure provides an engineered immune cell population comprising certain levels of biomarker-positive cells. In one embodiment, the engineered immune cell population comprises 65% or less HLA-DR+ cells and/or 30% or less TIGIT+ cells. In some embodiments, the engineered immune cell population with biomarker-positive cells comprises an exogenous nucleic acid sequence. In other embodiments, (i) the exogenous nucleic acid sequence comprises a chimeric antigen receptor (CAR) nucleic acid sequence or (ii) the exogenous nucleic acid sequence further comprises one or more nucleic acid sequences selected from the group consisting of a chimeric antigen receptor (CAR), a transmembrane domain nucleic acid sequence, a costimulatory domain nucleic acid sequence and a signaling domain nucleic acid sequence. In another embodiment, the exogenous nucleic acid sequence is expressed as a single transcript. In a further embodiment, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one additional aspect, the present disclosure provides methods of manufacturing immune cells with improved in vitro functionality. In some embodiments, the method comprises a step of detecting a level of HLA-DR expression in an immune cell population to provide a detected level of HLA-DR expression. In some embodiments, the detecting comprises detecting a protein level of HLA-DR using flow cytometry (FACS), an Enzyme-Linked Immunosorbent Assay (ELISA), an immunoblotting assay, an immunofluorescence assay, or an immunochemistry (IHC) assay. The method can further comprise a step of modifying the immune cell population to express an exogenous nucleic acid sequence, thereby providing an engineered immune cell population. In some embodiments, the engineered immune cell population comprises, exhibits, shows, or has improved in vitro functionality as compared to (i) an additional engineered immune cell population that originated from an additional immune cell population having a higher level of HLA-DR expression than the detected level, (ii) an additional engineered immune cell population originated from an additional immune cell population having a higher level of HLA-DR expression than the detected level, or (iii) an additional engineered immune cell population that was originated from an additional immune cell population having a higher level of HLA-DR expression than the detected level. The method may further comprise detecting different levels of expression for different biomarkers and combinations thereof. In other embodiments, the detected level indicates HLA-DR is expressed in less than 65% of immune cells of the immune cell population. In another embodiment, the lower level is more than 65% of immune cells of the additional immune cell population. In one embodiment, the modifying step further comprises reducing or eliminating expression or activity of an endogenous gene.

In some embodiments, the exogenous nucleic acid sequence comprises a chimeric antigen receptor (CAR) nucleic acid sequence. The exogenous nucleic acid sequence may further comprise one or more nucleic acid sequences selected from the group consisting of a transmembrane domain nucleic acid sequence, a costimulatory domain nucleic acid sequence and a signaling domain nucleic acid sequence. In other embodiments, the exogenous nucleic acid sequence is expressed as a single transcript. In some embodiments, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one embodiment, the improved in vitro functionality of the engineered immune cell population comprises, exhibits, shows, or has improved in vitro cytotoxicity. The improved in vitro functionality may comprise improved in vitro cytotoxicity. In some embodiments, the cytotoxicity is demonstrated by an in vitro killing assay. In one embodiment, the cytotoxicity is demonstrated by in vitro killing assay that comprises killing of cells that express a target of the CAR. In other embodiments, the in vitro killing assay is a long-term killing assay (LTKA) or a short-term killing assay (STKA).

In other embodiments, the immune cell population is obtained from or derived from a donor prior to the detecting step. The donor may be a healthy donor or a patient in need of treatment. In one embodiment, the patient is a patient in need of treatment with an autologous cell therapy. The autologous cell therapy may comprise the engineered immune cell population.

In another aspect, the present disclosure provides methods for selecting a donor immune cell population for engineering. In one embodiment, the method comprises a step of detecting a first level of HLA-DR expression in a first immune cell population to provide a first detected level of HLA-DR. In another embodiment, the method comprises a step of detecting a second level of HLA-DR expression in a second immune cell population to provide a second detected level of HLA-DR. In one other embodiment, the second detected level is greater than the first detected level. The method can comprise selecting the first immune cell population for engineering. In some embodiments, the method may further comprise discarding the second cell population and/or preserving the first cell population. In another embodiment, the detecting steps comprise detecting a protein level of HLA-DR using flow cytometry (FACS), an Enzyme-Linked Immunosorbent Assay (ELISA), an immunoblotting assay, an immunofluorescence assay, or an immunochemistry (IHC) assay. In other embodiments, the first detected level indicates that HLA-DR is expressed in less than 65% of immune cells of the immune cell population. In another embodiment, the second detected level indicates that HLA-DR is expressed in more than 65% of immune cells of the immune cell population.

In one embodiment, the method can further comprise a step of modifying the first immune cell population to express an exogenous nucleic acid sequence, thereby providing an engineered immune cell population. In some embodiments, the engineered immune cell population comprises, exhibits, shows, or has improved in vitro functionality as compared to an additional engineered immune cell population that originated from (or was originated from) the second immune cell population. The method may further comprise detecting different levels of expression for different biomarkers and combinations thereof. In one embodiment, the modifying step further comprises reducing or eliminating expression or activity of an endogenous gene.

In some embodiments, the exogenous nucleic acid sequence comprises a chimeric antigen receptor (CAR) nucleic acid sequence. The exogenous nucleic acid sequence may further comprise one or more nucleic acid sequences selected from the group consisting of a transmembrane domain nucleic acid sequence, a costimulatory domain nucleic acid sequence and a signaling domain nucleic acid sequence. In other embodiments, the exogenous nucleic acid sequence is expressed as a single transcript. In some embodiments, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one embodiment, the improved in vitro functionality of the engineered immune cell population comprises improved in vitro cytotoxicity. The improved in vitro functionality may comprise improved in vitro cytotoxicity. In some embodiments, the cytotoxicity is demonstrated by an in vitro killing assay. In one embodiment, the cytotoxicity is demonstrated by in vitro killing assay that comprises killing of cells that express a target of the CAR. In other embodiments, the in vitro killing assay is a long-term killing assay or a short-term killing assay.

In other embodiments, the immune cell population is obtained from or derived from a donor prior to the detecting step. The donor may be a healthy donor or a patient in need of treatment. In one embodiment, the patient is a patient in need of treatment with an autologous cell therapy. The autologous cell therapy may comprise the engineered immune cell population.

In a further aspect, the present disclosure provides methods for manufacturing immune cells with improved in vitro functionality. In one embodiment, the method comprises a step of modifying an immune cell population to express an exogenous nucleic acid sequence, thereby providing an engineered immune cell population. The method may further comprise depleting HLA-DR-positive engineered immune cells from the engineered immune cell population to provide an HLA-DR-depleted engineered immune cell population. In another embodiment, the HLA-DR-depleted engineered immune cell population comprises, exhibits, shows, or has improved in vitro functionality as compared to an engineered immune cell population that has not been depleted of HLA-DR-positive engineered immune cells. In one additional embodiment, the method further comprises depleting additional immune cells from the engineered immune cell population. In other embodiments, the additional immune cells express one or more of TIGIT, CD16, and CD56. In a further embodiment, the HLA-DR-depleted and TIGIT−, CD16−, or CD56-depleted engineered immune cell population comprises, exhibits, shows, or has improved in vitro functionality as compared to an engineered immune cell population that has not been depleted of HLA-DR-positive and TIGIT−, CD16- or CD56-positive immune cells. In another embodiment, the modifying step further comprises reducing or eliminating expression or activity of an endogenous gene. In one other embodiment, the depleting comprises a flow cytometry (FACS) method. In another embodiment, the method further comprises detecting a level of HLA-DR expression in the HLA-DR-depleted engineered immune cell population and/or detecting a level of TIGIT, CD16, and/or CD56 in the TIGIT−, CD16−, and/or CD56-depleted engineered immune cell population.

In some embodiments, the exogenous nucleic acid sequence comprises a chimeric antigen receptor (CAR) nucleic acid sequence. The exogenous nucleic acid sequence may further comprise one or more nucleic acid sequences selected from the group consisting of a transmembrane domain nucleic acid sequence, a costimulatory domain nucleic acid sequence and a signaling domain nucleic acid sequence. In other embodiments, the exogenous nucleic acid sequence is expressed as a single transcript. In some embodiments, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In other embodiments, the immune cell population is obtained from or derived from a donor prior to the detecting step. The donor may be a healthy donor or a patient in need of treatment. In one embodiment, the patient is a patient in need of treatment with an autologous cell therapy. The autologous cell therapy may comprise the engineered immune cell population.

In one other aspect, the present disclosure provides a chimeric antigen receptor T (CAR-T) cell populations. In one embodiment, the CAR-T cell population is a cell population in which HLA-DR is expressed at a first level and the CAR-T cell population has improved in vitro functionality as compared to a CAR-T cell population in which HLA-DR is expressed at a second level. In other embodiments, the first level is lower than the second level. In one other embodiment, the first level is more than 75% of the CAR-T cell population and/or wherein the second level is less than 75% of the CAR-T cell population.

In another embodiment, the CAR-T cell population has an exogenous nucleic acid sequence comprising a chimeric antigen receptor (CAR) nucleic acid sequence. The exogenous nucleic acid sequence may further comprise one or more nucleic acid sequences selected from the group consisting of a transmembrane domain nucleic acid sequence, a costimulatory domain nucleic acid sequence and a signaling domain nucleic acid sequence. In other embodiments, the exogenous nucleic acid sequence is expressed as a single transcript. In some embodiments, the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

In one embodiment, the improved in vitro functionality of the CAR-T cell population comprises improved in vitro cytotoxicity. The improved in vitro functionality may comprise improved in vitro cytotoxicity. In some embodiments, the cytotoxicity is demonstrated by an in vitro killing assay. In one embodiment, the cytotoxicity is demonstrated by in vitro killing assay that comprises killing of cells that express a target of the CAR. In other embodiments, the in vitro killing assay is a long-term killing assay or a short-term killing assay.

In an additional aspect, the preset disclosure provides a kit or an article of manufacture for in vitro functionality analysis of cell populations, e.g., donor cell populations and/or engineered cell populations. In one embodiment, the kit comprises an anti-HLA-DR binding agent. The kit may further comprise instructions to use the binding agent to detect a level of HLA-DR expression in the cell population. In other embodiments, the kit further comprises one or more additional binding agents to detect one or more of TIGIT, CD16, and CD56. The kit may further comprise instructions to use the binding agent(s) to detect a level of expression of one or more of TIGIT, CD16, CD56, CCR7, CD27, CD45RA, and any combination thereof in the cell population. In one embodiment, the kit further comprises reagents for measuring in vitro cytotoxicity of a CAR T cell engineered from the cell population. The binding agent(s) may be an antigen binding molecule, which may be an antibody or fragment thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1B depicts associations between in vitro CAR T cell functionality (long-term killing assay or LTKA) and certain biomarkers at the end of CAR-T cell manufacturing. FIG. 1C-1D depicts associations between in vitro CAR T cell functionality (long-term killing assay or LTKA) and other cell attributes (cell fitness and cytokine secretion).

FIG. 2A-2B depicts an observed negative correlation between the percentage of less differentiated T cells in both the starting material (FIG. 2A) and the CAR T cell product (FIG. 2B) and age, with older donors having less stem/central memory T cells than younger donors.

FIG. 3A-3B demonstrates that patient-derived CAR T cells also tended to have a lower percentage of Tscm at the end of culture compared to CAR T cells generated from healthy donor material, highlighting the limited fitness of disease donor T cells (FIG. 3A—starting material and FIG. 3B—after CAR T manufacturing).

FIG. 4A-4B demonstrates that T cells from younger donors have a younger T cell phenotype, lower expression of exhaustion marker (e.g., HLA-DR and TIGIT) and better in vitro cytotoxicity.

FIG. 5 shows an exemplary protocol for isolating donor cells (e.g., PBMCs), biomarker profiling, activating, transducing, transfecting, depleting, expanding, and harvesting T cells from the isolated donor cells.

### DETAILED DESCRIPTION

The instant disclosure relates to methods and reagents for analyzing cell populations, such as donor cell populations and/or engineered cell populations, to identify candidates for manufacturing of engineered cell populations and/or improve the manufacturing process for engineered cell populations.

Provided herein are methods and reagents for analysis and/or characterization of cell populations, such as donor cell populations, including without limitation donor immune cell populations, for example peripheral blood mononuclear cells (PBMCs), for use in selecting donor cells for manufacturing of engineered cell populations, e.g., engineered immune cell populations, such as CAR T cell populations. The methods and reagents disclosed herein allow for the identification of donor cell populations based on one or more attributes, which if present, result in improved in vitro functionality in engineered cell populations that are derived from the donor cells as compared to donor cell populations that do not have the one or more attributes. Also provided herein are processes, workflows, kits, articles of manufacture and reagents that allow reliable and convenient analysis of critical attributes of donor cell populations.

In addition, the instant disclosure provides methods and reagents for analysis and/or characterization of engineered cell populations, such as engineered immune cell populations, including without limitation CAR-T cell populations, for use in the manufacturing process of such engineered cell populations. The methods and reagents disclosed herein allow for the identification of one or more attributes in engineered cell populations, which if present, result in improved in vitro functionality in the engineered cell population as compared to an engineered immune cell population that does not have the one or more attributes. Also provided herein are processes, workflows, kits, articles of manufacture and reagents that allow reliable and convenient analysis of critical attributes of engineered cell populations.

As used herein, the terms “a” and “an” are used to mean one or more. For example, a reference to “a cell” or “an antibody” means “one or more cells” or “one or more antibodies.”

As used herein, the term “biomarker-depleted” refers to a donor cells from a donor cell population or a donor cell population where an unwanted subset of cells expressing one or more biomarkers, e.g., cell surface protein markers, has been separated from the original donor cell population. The one or more biomarkers include, without limitation, HLA-DR, TIGIT, CD16, CD56, and any combination thereof. A biomarker-depleted donor cell population, e.g., an HLA-DR-depleted and/or TIGIT-depleted donor cell population, means a population of donor cells that comprises fewer cells expressing one or more biomarkers than a donor cell population which has not been depleted of the one or more biomarkers, according to the methods described herein. For example, a population of HLA-DR-depleted cells (or HLA-DR-depleted cells) can comprise 10%, 9%, 8%, 7%, 6%, 5%, 4%, 3%, 2%, 1% or less than 1% of cells expressing HLA-DR.

As used herein, the term “labeling agent” generally refers to an agent capable of interacting with a component of a cell including, without limitation, the cell membrane, a molecule on and/or within the cell, an intracellular molecule of the cell, etc. The interaction between the agent and the cell component may be a covalent interaction or a non-covalent interaction, a reversible interaction, or an irreversible interaction. The labeling agent may be specific to the cell component including, without limitation, a biological molecule of the cell (e.g., a polypeptide, a nucleic acid, a lipid, etc.). In some embodiments, the labeling agent may be an agent having specificity to a biological target, such as an antibody or an antibody fragment. In one embodiment, the labeling agent is an agent having specificity to a cell surface molecule, e.g., a cell surface or cell membrane protein. In some cases, the labelling agent can include one or more detectable labels. In some embodiments, the labeling agent comprises an antibody, optionally conjugated with a detectable label.

In some embodiments, the detectable label is selected from the group consisting of a fluorescent label, a photochromic compound, a proteinaceous fluorescent label, a molecule capable of a colorimetric reaction, a magnetic label, a radiolabel, an oligonucleotide label, and a hapten. In some embodiments, the fluorescent label is selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoeryhring (BPE), mCherry, HcRed1, Katusha, P3, Peridinin Chlorophyll (PerCP), mKate (TagFP635), TurboFP635, mPlum, and mRaspberry. In some embodiments, the one or more labeling agents are used for flow cytometry. The one or more labels may be directly or indirectly coupled to or conjugated to labelling agents. For an indirect format, the one or more labels may be coupled to or conjugated to a molecule that can bind to the labeling agent. For example, the label may be conjugated to an oligonucleotide sequence that is complementary to another oligonucleotide sequence from an oligonucleotide conjugated to the labeling agent (e.g., an antibody conjugated to an oligonucleotide). Labels may also be used with the methods and compositions of the present disclosure in the context of binding agents, such as secreted molecule binding agents, e.g., secreted cytokine binding agents.

### Attributes of Cell Populations

In one aspect, the instant disclosure concerns the detection, identification, and/or selection of cells from a cell population, such as donor cells from a donor cell population, that have desired attributes prior to modification of such cells to manufacture engineered cells, e.g., prior to modification of donor cells to manufacture CAR T cell products. The methods and reagents disclosed herein allow for the identification of cell populations, e.g., donor cell populations, based on one or more attributes, which if present, correlate to improved in vitro functionality for engineered cell populations (e.g., engineered immune cell populations) that are derived from the cells (e.g., donor cells) as compared to cell populations that do not have the one or more attributes. One or more attributes may be used to screen cell populations (e.g., donor cell populations) including, without limitation, detection of the presence or absence of one or more biomarkers as described herein. In vitro functionality may be assessed in different ways including, without limitation, cytotoxicity, cytokine secretion profiling, and cell fitness (e.g., mitochondrial fitness). In addition, engineered cells (e.g., engineered immune cells, such as CAR-T cells) derived from donor cells may be subjected to the same detection, identification, and/or selection methods based on the same or different attributes that were analyzed in the donor cells.

### Biomarker Detection

In one aspect, the present disclosure provides methods and reagents to screen or analyze cells from a cell population (e.g., donor cells of a donor cell population and/or engineered cells of an engineered cell population) for a percentage of cells from the cell population that express one or more biomarkers. In one embodiment, the biomarker is one or more of the following: T cell immunoreceptor with Ig and immunoreceptor tyrosine-based inhibitory motif (ITIM) domains (TIGIT), human leukocyte antigen-DR isotype (HLA-DR), CD16, CD56, CD27, Chemokine Receptor 7 (CCR7), CD45RA, and any combination thereof. In one other embodiment, the cells are characterized by (a) a percentage of one or more of the following biomarkers: TIGIT, HLA-DR, CD16, CD56, and any combination thereof and/or (b) a percentage of one or more of the following biomarkers: CD27, CCR7, CD45RA, and any combination thereof.

In other embodiments, the detected percentage of cells expressing a biomarker described herein (e.g., HLA-DR, TIGIT, CD16, CD56, CD27, CCR7, CD45RA, and any combination thereof) is between about 0% and about 100% of the cell population being analyzed. In other embodiments, the percentage of cells expressing a biomarker is about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In one aspect, the methods, compositions, cell populations and kits concern the detection of, identification of, and/or screening of cells (donor cells and/or engineered cells) from cell populations (e.g., donor cell populations and/or engineered cell populations) for a level of HLA-DR expression as a percentage of the cell population being tested. Certain percentages for certain biomarkers in certain cell populations can be predictive of the degree of in vitro functionality for engineered immune cells from an engineered immune cell population, e.g., a CAR-T cell population, derived from donor cells of a donor cell population.

In one embodiment, the donor cells of a donor cell population are obtained from a human donor who is a younger donor. A younger donor is a donor with an age of about 30 year or less. In other embodiments, the younger donor's age about 18, about 19, about 20, about 21, about 22, about 23, about 24, about 25, about 26, about 27, about 28, about 29, or about 30 years old. In one other embodiment, the donor is between about 18 and about 30 years old, between about 18 and about 29 years old, between about 19 and about 30 years old, or between about 19 and about 29 years old. In one other embodiment, the expression of HLA-DR in a donor cell population from the younger donor is lower than the expression of HLA-DR in a donor cell population from a donor with an age greater than 30 years old.

In one embodiment, the level indicates that HLA-DR is expressed in less than 65% of cells in the cell population. In another embodiment, the <65% of cells correlates to improved in vitro functionality, e.g., cytotoxicity, in a cell population, such as an engineered immune cell population. In one other embodiment, the level indicates that HLA-DR is expressed in more than 65% of cells in the cell populations. The <65% population exhibits stronger in vitro functionality (e.g., cytotoxicity) than the >65% population. In other embodiments, the percentage of HLA-DR-expressing cells that correlate to improved in vitro functionality, e.g., cytotoxicity, in the cell population may be detected within a range of percentages. In one embodiment, the <65% percentage range of HLR-DR-expressing cells may be between about 30% and about 35%, about 35% and about 40%, about 40% and about 45%, about 45% and about 50%, or about 55% and about 60%. In another embodiment, the <65% level of HLR-DR-expressing cells is about 60%, about 61%, about 62%, about 63%, about 64%, or about 64.5%.

In one other embodiment, the percentage range of HLR-DR-expressing cells may be detected as being 65% or greater, which is less preferred than less than 65%. In another embodiment, such cells may express HLA-DR at a level between about 65% and about 90%, about 70% and about 85%, about 75% and about 80%, about 65% and about 70%, about 65% and about 75%, about 70% and about 75%, about 85% and about 90%, about 80% and about 90%, or about 75% and about 90%. In one embodiment, the cells are not used in the methods described herein if the detected HLA-DR level is greater than 90%.

Additional levels of additional biomarkers may be analyzed. In another embodiment, a level of TIGIT expression as a percentage of the cell population being tested is detected. In one embodiment, the level indicates that TIGIT is expressed in less than 30% of cells in the cell population. In one embodiment, the <30% of TIGIT-expressing cells correlates to improved in vitro functionality, e.g., cytotoxicity, in a cell population, such as an engineered immune cell population. In one other embodiment, the level indicates that TIGIT is expressed in more than 30% of cells in the cell populations. The <30% population exhibits stronger in vitro functionality (e.g., cytotoxicity) than the >30% population. In other embodiments, the percentage of TIGIT-expressing cells that correlate to improved in vitro functionality, e.g., for engineered immune cells, in a cell population may be detected within a range of percentages. In one embodiment, the <30% percentage range of TIGIT-expressing cells may be between about 1% and about 5%, about 5% and about 10%, about 10% and about 15%, about 15% and about 20%, or about 20% and about 25%. In another embodiment, the <30% level of TIGIT-expressing cells is about 26%, about 27%, about 28%, about 29%, or about 29.5%.

In one other embodiment, the percentage range of TIGIT-expressing cells may be detected as being 30% or greater, which is less preferred than less than 30%. In another embodiment, such cells may express TIGIT at a level between about 30% and about 55%, about 35% and about 50%, about 40% and about 45%, about 30% and about 35%, about 30% and about 40%, about 30% and about 45%, about 50% and about 55%, about 45% and about 55%, or about 50% and about 55%. In one embodiment, the cells are not used in the methods described herein if the detected HLA-DR level is greater than 55%.

In one additional embodiment, a level of CD16 expression as a percentage of the cell population being tested is detected. In one embodiment, the level indicates that CD16 is expressed in less than 4% of cells in the cell population. In another embodiment, the <4% of cells correlates to improved in vitro functionality, e.g., cytotoxicity, in a cell population, such as an engineered immune cell population. In one other embodiment, the level indicates that CD16 is expressed in more than 4% of cells in the cell populations. The <4% population exhibits stronger in vitro functionality (e.g., cytotoxicity) than the >4% population. In other embodiments, the percentage of CD16-expressing cells that correlate to improved in vitro functionality, e.g., cytotoxicity, in the cell population may be detected within a range of percentages. In one embodiment, the <4% percentage range of CD16-expressing cells may be about 0.5%, about 1%, about 1.5%, about 2%, about 2.5%, about 3%, about 3.5%, or about 3.75%.

In another embodiment, the percentage range of CD16-expressing cells may be detected as being greater than 4%, which is less preferred than less than 4%. In one other embodiment, such cells may express CD16 at a level between about 4% and about 10%, about 5% and about 9%, about 6% and about 8%, about 4% and about 5%, about 4% and about 6%, about 4% and about 7%, about 4% and about 8%, about 4% and about 9%, or about 9% and about 10%, about 8% and about 10%, about 7% and about 10%, about 6% and about 10%, or about 5% and about 10%. In one embodiment, the cells are not used in the methods described herein if the detected CD16 level is greater than 10%.

In one additional embodiment, a level of CD56 expression as a percentage of the cell population being tested is detected. In one embodiment, the level indicates that CD56 is expressed in less than 15% of cells in the cell population. In another embodiment, the <15% of cells correlates to improved in vitro functionality, e.g., cytotoxicity, in a cell population, such as an engineered immune cell population. In one other embodiment, the level indicates that CD56 is expressed in more than 15% of cells in the cell populations. The <15% population exhibits stronger in vitro functionality (e.g., cytotoxicity) than the >15% population. In other embodiments, the percentage of CD56-expressing cells that correlate to improved in vitro functionality, e.g., cytotoxicity, in the cell population may be detected within a range of percentages. In one embodiment, the <15% percentage range of CD56-expressing cells may be between about 1% and about 5% or about 5% and about 10%. In another embodiment, the <15% level of CD56-expressing cells is about 11%, about 11.5%, about 12%, about 12.5%, about 13%, about 13.5%, about 14%, about 14.5% or about 14.75%.

In another embodiment, the percentage range of CD56-expressing cells may be detected as being greater than 15%, which is less preferred than less than 15%. In another embodiment, such cells may express CD56 at a level between about 15% and about 20%, about 16% and about 19%, about 17% and about 18%, about 15% and about 16%, about 15% and about 17%, about 15% and about 18%, about 15% and about 19%, about 19% and about 20%, or about 18% and about 20%, about 17% and about 20%, about 18% and about 20%, or about 19% and about 20%. In one embodiment, the cells are not used in the methods described herein if the detected CD56 level is greater than 20%.

In one additional embodiment, a level of CCR7 expression as a percentage of the cell population being tested is detected. In one embodiment, the level indicates that CCR7 is expressed in more than 30% of cells in the cell population. In another embodiment, the >30% of cells correlates to improved in vitro functionality, e.g., cytotoxicity, in a cell population, such as an engineered immune cell population. In one other embodiment, the level indicates that CCR7 is expressed in less than 30% of cells in the cell populations. The >30% population exhibits stronger in vitro functionality (e.g., cytotoxicity) than the <30% population. In other embodiments, the percentage of CCR7-expressing cells that correlate to improved in vitro functionality, e.g., cytotoxicity, in the cell population may be detected within a range of percentages. In one embodiment, the >30% percentage range of CCR7-expressing cells may be between about 35% and about 40%, about 40% and about 45%, about 45% and about 50%, about 50% and about 55%, about 55% and about 60%, about 60% and about 65%, or about 65% and about 70%. In another embodiment, the >30% level of CCR7-expressing cells is about 30.5%, about 31%, about 32%, about 33%, or about 34%.

In another embodiment, the percentage range of CCR7-expressing cells may be detected as being less than 30%, which is less preferred than greater than 30%. In one other embodiment, the <30% percentage range of CCR7-expressing cells may be between about 15% and about 30%, about 20% and about 25%, about 15% and about 20%, about 20% and about 25%, about 15% and about 25%, about 25% and about 30%, or about 20% and about 30%. In one embodiment, the cells are not used in the methods described herein if the detected CCR7 level is less than 15%.

In one additional embodiment, a level of CD27 expression as a percentage of the cell population being tested is detected. In one embodiment, the level indicates that CD27 is expressed in more than 55% of cells in the cell population. In another embodiment, the >55% of cells correlates to improved in vitro functionality, e.g., cytotoxicity, in a cell population, such as an engineered immune cell population. In one other embodiment, the level indicates that CD27 is expressed in less than 55% of cells in the cell populations. The >55% population exhibits stronger in vitro functionality (e.g., cytotoxicity) than the <55% population. In other embodiments, the percentage of CD27-expressing cells that correlate to improved in vitro functionality, e.g., cytotoxicity, in the cell population may be detected within a range of percentages. In one embodiment, the >55% percentage range of CD27-expressing cells may be between about 60% and about 65%, about 65% and about 70%, about 70% and about 75%, about 75% and about 80%, about 80% and about 85%, about 85% and about 90%, or about 90% and about 95%. In another embodiment, the >55% level of CD27-expressing cells is about 55.5%, about 56%, about 57%, about 58%, or about 59%.

In another embodiment, the percentage range of CD27-expressing cells may be detected as being less than 55%, which is less preferred than greater than 55%. In one other embodiment, such cells may express CD27 at a level between about 30% and about 55%, about 35% and about 50%, about 35% and about 45%, about 30% and about 35%, about 30% and about 40%, about 30% and about 45%, about 30% and about 50%, about 50% and about 55%, about 45% and about 55%, about 40% and about 55%, or about 35% and about 55%. In one embodiment, the cells are not used in the methods described herein if the detected CD27 level is less than 30%.

In one additional embodiment, a level of CD45RA expression as a percentage of the cell population being tested is detected. In one embodiment, the level indicates that CD45RA is expressed in more than 70% of cells in the cell population. In another embodiment, the >70% of cells correlates to improved in vitro functionality, e.g., cytotoxicity, in a cell population, such as an engineered immune cell population. In one other embodiment, the level indicates that CD45RA is expressed in less than 70% of cells in the cell populations. The >70% population exhibits stronger in vitro functionality (e.g., cytotoxicity) than the <70% population. In other embodiments, the percentage of CD45RA-expressing cells that correlate to improved in vitro functionality, e.g., cytotoxicity, in the cell population may be detected within a range of percentages. In one embodiment, the >70% percentage range of CD45RA-expressing cells may be between about 75% and about 80%, about 80% and about 85%, about 85% and about 90%, or about 90% and about 95%. In another embodiment, the >70% level of CD45RA-expressing cells is about 70.5%, about 71%, about 72%, about 73%, or about 74%.

In another embodiment, the percentage range of CD45RA-expressing cells may be detected as being less than 70%, which is less preferred than greater than 70%. In one other embodiment, such cells may express CD45RA at a level between about 50% and about 70%, about 55% and about 65%, about 55% and about 60%, about 50% and about 55%, about 50% and about 60%, about 50% and about 65%, about 65% and about 70%, about 60% and about 70%, or about 55% and about 70%. In one embodiment, the cells are not used in the methods described herein if the detected CD45RA level is less than 50%.

Tables 1-10 represent non-limiting examples of different biomarker profiles for cells of a cell population (e.g., donor cells of a donor cell population and/or engineered cells of an engineered cell population) that can be detected with the methods described herein. The asterisk (*) indicates that the biomarker can be detected in cells of a cell population at the following percentages.
HLA-DR: <65% or between 65-90%;
TIGIT: <30% or between 30-55%;
CD16: <4% or between 4-10%;
CD56: <15% or between 15-20%;
CCR7: >30% or between 15-30%;
CD27: >55% or between 30-55%; and
CD45RA: >70% or between 50-70%.

For example, cell biomarker profile #3 in Table 1 correspond to cells (e.g., donor cells of a donor cell population and/or engineered cells of an engineered cell population) with the following percentages of expression for the following biomarkers: HLA-DR: <65% or between 65-90% and TIGIT: <30% or between 30-55%. In another example, cell biomarker profile #12 in Table 1 correspond to cells (e.g., donor cells of a donor cell population and/or engineered cells of an engineered cell population) with the following percentages of expression for the following biomarkers: TIGIT: <30% or between 30-55%.

TABLE 1

| | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| HLA-DR | * | * | * | * | * | * | * | | | | | | | | |
| TIGIT | * | * | * | | | | | * | * | * | * | * | | | |
| CD16 | * | * | | | * | | | * | * | | | | * | * | |
| CD56 | * | | | | * | * | | * | | | * | | * | | * |

TABLE 2

| | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|
| CD27 | * | * | * | * | * | * | * | * | * | * | * | * | * | * | * |
| HLA-DR | * | * | * | * | | | | | | | | * | * | * | |
| TIGIT | * | * | * | | | * | * | * | | | | | * | | |
| CD16 | * | * | | | | * | * | | * | * | | | | | |
| CD56 | * | | | | | * | | | * | | * | * | * | * | * |

TABLE 3

| | 31 | 32 | 33 | 34 | 35 | 36 | 37 | 38 | 39 | 40 | 41 | 42 | 43 | 44 | 45 |
|---|---|---|---|---|---|---|---|---|---|---|---|---|---|---|---|
| CCR7 | * | * | * | * | * | * | * | * | * | * | * | * | * | * | * |
| HLA-DR | * | * | * | * | | | | | | | | * | * | * | |
| TIGIT | * | * | * | | | * | * | * | | | | | * | | |
| CD16 | * | * | | | | * | * | | * | * | | | | | |
| CD56 | * | | | | | * | | | * | | * | * | * | * | * |

TABLE 4

| | 46 | 47 | 48 | 49 | 50 | 51 | 52 | 53 | 54 | 55 | 56 | 57 | 58 | 59 | 60 |
|---|---|---|---|---|---|---|---|---|---|---|---|---|---|---|---|
| CD45RA | * | * | * | * | * | * | * | * | * | * | * | * | * | * | * |
| HLA-DR | * | * | * | * | | | | | | | | * | * | * | |
| TIGIT | * | * | * | | | * | * | * | | | | | * | | |
| CD16 | * | * | | | | * | * | | * | * | | | | | |
| CD56 | * | | | | | * | | | * | | * | * | * | * | * |

TABLE 5

| | 61 | 62 | 63 | 64 | 65 | 66 | | 67 | 68 | 69 | 70 | 71 | 72 |
|---|---|---|---|---|---|---|---|---|---|---|---|---|---|
| TIGIT | * | * | * | * | * | * | HLA-DR | * | * | * | * | * | * |
| CD27 | * | * | * | | | | CD27 | * | * | * | | | |
| CCR7 | * | * | | * | * | | CCR7 | * | * | | * | * | |
| CD45RA | * | | | * | | * | CD45RA | * | | | * | | * |

TABLE 6

| | 73 | 74 | 75 | 76 | 77 | 78 | | 79 | 80 | 81 | 82 | 83 | 84 |
|---|---|---|---|---|---|---|---|---|---|---|---|---|---|
| CD16 | * | * | * | * | * | * | CD56 | * | * | * | * | * | * |
| CD27 | * | * | * | | | | CD27 | * | * | * | | | |
| CCR7 | * | * | | * | * | | CCR7 | * | * | | * | * | |
| CD45RA | * | | | * | | * | CD45RA | * | | | * | | * |

TABLE 7

| | 85 | 86 | 87 | 88 | 89 | 90 | | 91 | 92 | 93 | 94 | 95 | 96 |
|---|---|---|---|---|---|---|---|---|---|---|---|---|---|
| TIGIT | * | * | * | * | * | * | TIGIT | * | * | * | * | * | * |
| HLA-DR | * | * | * | * | * | * | CD16 | * | * | * | * | * | * |
| CD27 | * | * | * | | | | CD27 | * | * | * | | | |
| CCR7 | * | * | | * | * | | CCR7 | * | * | | * | * | |
| CD45RA | * | | | * | | * | CD45RA | * | | | * | | * |

TABLE 8

| | 97 | 98 | 99 | 100 | 101 | 102 | | 103 | 104 | 105 | 106 | 107 | 108 |
|---|---|---|---|---|---|---|---|---|---|---|---|---|---|
| TIGIT | * | * | * | * | * | * | HLA-DR | * | * | * | * | * | * |
| CD56 | * | * | * | * | * | * | CD16 | * | * | * | * | * | * |
| CD27 | * | * | * | | | | CD27 | * | * | * | | | |
| CCR7 | * | * | | * | * | | CCR7 | * | * | | * | * | |
| CD45RA | * | | | * | | * | CD45RA | * | | | * | | * |

TABLE 9

| | 109 | 110 | 111 | 112 | 113 | 114 | | 115 | 116 | 117 | 118 | 119 | 120 |
|---|---|---|---|---|---|---|---|---|---|---|---|---|---|
| HLA-DR | * | * | * | * | * | * | CD56 | * | * | * | * | * | * |
| CD56 | * | * | * | * | * | * | CD16 | * | * | * | * | * | * |
| CD27 | * | * | * | | | | CD27 | * | * | * | | | |
| CCR7 | * | * | | * | * | | CCR7 | * | * | | * | * | |
| CD45RA | * | | | * | | * | CD45RA | * | | | * | | * |

TABLE 10

| | 121 | 122 | 123 | 124 | 125 | 126 |
|---|---|---|---|---|---|---|
| CD27 | * | * | * | * | | |
| CCR7 | * | * | | | * | |
| CD45RA | * | | * | | | * |

The detection of one or more biomarkers of cells from a cell population (e.g., donor cells from a donor cell population and/or engineered cells from an engineered cell population) may be performed using different methodologies including, without limitation, flow cytometry, fluorescence activated cell sorting, (FACS) flow cytometry, an Enzyme-Linked Immunosorbent Assay (ELISA), an Enzyme-linked immuno-filtration assay (ELIFA), an immunoblotting assay, an immunofluorescence assay, an immunochemistry (IHC) assay, western blot analysis, and immunoprecipitation, molecular binding assays.

Detection using Flow cytometry. Instruments for particle analysis, e.g., flow and scanning cytometers, allow individual particles or cells (e.g., single donor cells from a donor cell population and/or single engineered cells from an engineered cell population) to be characterized using optical parameters such as light scatter and fluorescence. For example, a flow cytometer allows an aqueous suspension containing individual particles (e.g., beads comprising analytes of interest) or cells to be passed by a detection region that exposes the suspension to an excitatory light source, such as one or more lasers, thereby enabling a user to measure the light scattering and fluorescence properties of the particles or cells. The labeling of particles or cells with one or more fluorescent dyes can facilitate detection. A plurality of different particles or cells may be subjected to flow cytometry for simultaneous detection by using different dyes that are spectrally distinct to label different particles or cells. In other embodiments, multiple photodetectors can be deployed to measure different scatter parameters and/or different spectrally distinct dyes. For example, one or more detectors may be configured to measure one or more sets of scatter parameters and one or more additional detectors may be configured to measure one or more distinct dyes, which would allow the generation of data comprising signals for each light scatter parameter and each fluorescence emission.

Flow cytometry parameters that are commonly measured include, without limitation, (i) the excitation light that is scattered by the particle or cell along a mostly forward direction, or forward scatter (FSC), (ii) the excitation light that is scattered by the particle or cell in a mostly sideways direction, or side scatter (SSC), and (ii) the light emissions from fluorescent molecules in one or more channels (range of frequencies) of the spectrum or by the fluorescent dye that is primarily detected in that channel. In one embodiment, different cell types from a donor cell population can be identified by the FSC, SSC, and fluorescence emissions resulting from labeling various cell-surface proteins on the donor cells with dye-labeled antibodies.

The data obtained from an analysis of particles or cells (e.g., single donor cells from a donor cell population and/or single engineered cells from an engineered cell population) by multi-color flow cytometry are multidimensional, wherein each cell corresponds to a point in a multidimensional space defined by the parameters measured. Populations of cells or particles are identified as clusters of points in the data space. The identification of clusters and, thereby, populations can be carried out manually by drawing a gate around a population displayed in one or more 2-dimensional plots, referred to as “scatter plots” or “dot plots,” of the data. Alternatively, clusters can be identified, and gates that define the limits of the populations, can be determined automatically. Flow cytometry is an important tool for the analysis and/or isolation of particle or cells (e.g., individual donor cells from a donor cell populations) and cellular analytes or constituents thereof. Therefore, it can be used in the context of donor cell analysis and/or isolation. In one embodiment, the present disclosure provides a method using a fluid stream to linearly space donor cells from a donor cell population such that they pass individually through a detection apparatus. A single cell from the donor cell population can be distinguished from other cells by their location in the fluid stream and the presence of detectable markers. As a result, a flow cytometer can be used to detect one or more biomarkers (as described herein) and/or generate a biomarker profile for a donor cell population and/or an engineered cell population. Such profiles may comprise a percentage of cells (as further described herein) expressing one or more biomarkers out of the total cell population that is being analyzed.

In one additional aspect, the instant disclosure provides a method of detecting cells from a cell population (e.g., donor cells from a donor cell population and/or engineered cells from an engineered cell population) by detecting a level of one or more cell surface biomarkers. In one embodiment, the detected level indicates that some of the cells (e.g., donor cells and/or engineered cells) express the one or more cell surface biomarkers. In other embodiments, the detected level indicates that some of the cells express a first biomarker and/or some of the cells express a second biomarker. In some embodiments, the cells express one or more cell surface markers HLA-DR, TIGIT, CD16, CD56, CCR7, CD27, CD45RA, or any combination thereof. In another aspect, the level of one or more biomarkers that is detected indicates the number of cells (e.g., donor cells and/or engineered cells) expressing the one or more biomarkers out of the cell population being analyzed (e.g., donor cell population and/or engineered cell population). Tables 1-10 above provide additional biomarker profiles for donor cells of a donor cell population.

In some embodiments, the method provides a quantitative measurement of donor cells from a donor cell population that do not express cell surface biomarkers, e.g., HLA-DR, TIGIT, CD16, CD56, CCR7, CD27, CD45RA, or any combination thereof. Flow cytometry can be used to quantify cells expressing or not expressing specific surface markers, or quantifying cells of a specific cell type, within a population of cells.

As described herein, flow cytometry is a method for quantifying components or structural features of cells primarily by optical means using certain labeling agents (as further described herein). Since different cell types can be distinguished by quantifying structural features, flow cytometry and cell sorting can be used to count and sort cells of different phenotypes in a mixture. A flow cytometry analysis involves two primary steps: 1) labeling selected cell types with one or more detectable labels or agents, and 2) determining the number of labeled cells relative to the total number of cells in the population. In some embodiments, the method of labeling cell types includes binding labeled antibodies to markers expressed by the specific cell type. The antibodies may be either directly labeled with a fluorescent compound or indirectly labeled using, for example, a fluorescent-labeled second antibody which recognizes the first antibody.

In one other aspect, the present disclosure utilizes flow cytometry to provide methods for generating a biomarker profile for a cell population (e.g., a donor cell population or an engineered cell population) based on the percentage of one or more biomarkers detected. In one embodiment, the method comprises providing a donor cell population, such as a donor immune cell population, that is suspected of comprising one or more biomarkers. In another embodiment, the method further comprises detecting a level of a first biomarker in the donor cell population, wherein the detected level of the first biomarker indicates that a percentage of the donor cell population expresses the first biomarker. In other embodiments, the first biomarker is HLA-DR. In other embodiments, the method further comprises detecting a level of a second biomarker in the donor cell population, wherein the detected level of the second biomarker indicates that a percentage of the donor cell population expresses the second biomarker. In one additional embodiment, the second biomarker is TIGIT. In some embodiments, the method comprises detecting levels of the first and second biomarker, wherein the detected levels indicate a percentage expression in the donor cell population of the first biomarker and the second biomarker. In other embodiments, one or more additional biomarkers, e.g., third, fourth, fifth, sixth, seventh, etc. biomarkers, are detected to determine whether the percentage of expression in donor cells of the donor cell population for one or more additional biomarkers.

In some embodiments, the methods for generating a biomarker profile further comprise steps to modify or select for modification a donor cell population that has been subjected to biomarker profile generation, such as by using flow cytometry. In one embodiment, the method further comprises modifying the donor cell population to express an exogenous nucleic acid sequence, thereby providing an engineered cell population. In additional embodiments, the engineered immune cell population comprises, exhibits, shows, or has improved in vitro functionality as compared to an additional engineered immune cell population that originated from (or was originated from) an additional immune cell population that expresses a percentage of the first biomarker (e.g., HLA-DR) and/or expresses a percentage of the second biomarker (e.g., TIGIT). The engineered immune cell population may be express a percentage of two or more biomarkers, e.g., two or more of HLA-DR, TIGIT, CD16, CD56, and any combination thereof. In other embodiments, the exogenous nucleic acid comprises a chimeric antigen receptor (CAR) nucleic acid sequence. The CAR sequence can include one or more nucleic acid sequences including, without limitation, a transmembrane domain nucleic acid sequence, a costimulatory domain nucleic acid sequence and a signaling domain nucleic acid sequence. In one embodiment, the exogenous nucleic acid sequence is expressed as a single transcript. In addition to or independent of the modification to express an exogenous nucleic acid sequence, the method may further comprise reducing or eliminating expression or activity of an endogenous gene, e.g., a T cell receptor gene (e.g., TCRα, TCRβ) and/or CD52 as further described herein.

Prior to engineering, immune cell populations as described herein may be obtained from and/or derived from a donor prior to any biomarker detection steps. In some embodiments, the donor is a healthy donor or a patient need of treatment. The patient in need of treatment may be in need of treatment with an autologous cell therapy. In one embodiment, the autologous cell therapy may comprise engineered immune cell populations that were derived from donor cells obtain from the patient.

As described herein, the engineered cell population having a detected biomarker profile is characterized by improved in vitro functionality, which can be improved in vitro cytotoxicity and/or improved in vitro cell fitness, e.g., in vitro mitochondrial fitness. Cytotoxicity can be demonstrated by an in vitro killing assay, such as an in vitro killing assay that comprises the killing of cells that express a target molecule that is recognized by the CAR. The in vitro killing assay may be an in vitro short-term killing assay (STKA) or an in vitro long-term killing assay (LTKA).

In one additional embodiment, the engineered immune cell population exhibits improved in vitro cytotoxicity as shown by an increased area under the curve or AUC. As used herein, the term “area under the curve” or “AUC” refers to a quantified measurement of both persistence (length of assay) and cytotoxicity (% killing) for LTKA. In the context of a STKA, “AUC” refers to a quantified measurement of cytotoxicity at different tumor burdens (distance between different effector-to-target ratio as 1). STKA and LTKA AUC can be measured as described in Example 2.

In other embodiments, the increased AUC is relative to an AUC observed for an engineered immune cell population derived from a non-healthy donor, e.g., a subject having a disease, such as cancer. In some embodiments, the AUC is a STKA AUC or a LTKA AUC. In other embodiments, the LTKA AUC is between about 749 and about 1217. In another embodiment, the LTKA AUC is between about 200 and about 1500, between about 225 and about 1475, between about 225 and about 1475, between about 250 and about 1450, between about 275 and about 1425, between about 300 and about 1400, between about 325 and about 1375, between about 325 and about 1350, between about 350 and about 1325, between about 350 and about 1300, between about 375 and about 1275, between about 400 and about 1250, between about 375 and about 1225, between about 400 and about 1250, between about 425 and about 1275, between about 450 and about 1200, between about 475 and about 1175, between about 500 and about 1150, between about 525 and about 1125, between about 550 and about 1100, between about 575 and about 1075, between about 600 and about 1050, or between about 625 and about 1025. In other embodiments, the LTKA AUC is between about 700 and about 1250, between about 725 and about 1250, between about 750 and about 1250, between about 775 and about 1250, between about 800 and about 1250, between about 825 and about 1250, between about 850 and about 1250, between about 875 and about 1250, between about 900 and about 1250, between about 925 and about 1250, between about 950 and about 1250, between about 975 and about 1250, between about 1000 and about 1250, between about 1025 and about 1250, between about 1050 and about 1250, between about 1075 and about 1250, between about 1100 and about 1250, between about 1125 and about 1250, between about 1150 and about 1250, between about 1175 and about 1250, between about 1200 and about 1250, or between about 1225 and about 1250. In some embodiments, the LTKA AUC is at least about 1100, at least about 1125, at least about 1150, at least about 1175, at least about 1200, at least about 1225, at least about 1250, at least about 1275, at least about 1300, at least about 1325, at least about 1350, at least about 1375, at least about 1400, at least about 1425, at least about 1450, at least about 1475, or at least about 1500.

In another embodiment, the AUC is a STKA AUC. In one embodiment, the STKA AUC is between about 209 and about 519.

In another embodiment, the in vitro functionality is in vitro cell fitness, e.g., mitochondrial fitness. Mitochondrial fitness can be measured by spare respiratory capacity (SRC) as described in Example 2. In one embodiment, the engineered immune cell population exhibits improved mitochondrial fitness as shown by an increased SRC. In other embodiments, the increased SRC is relative to an SRC observed for an engineered immune cell population derived from a non-healthy donor, e.g., a subject having a disease, such as cancer. In some embodiments, the SRC is between about 27 and about 59. In another embodiment, the SRC is about 25, about 30, about 35, about 40, about 45, about 50, about 55, about 60, about 65, about 70, about 75, about 80, about 85, about 90, about 95, or about 100.

Enrichment or depletion. In one other aspect, the instant disclosure provides methods and compositions for the enrichment and/or depletion of cell from a cell population (e.g., donor cells from a donor cell population and/or engineered cells from an engineered cell population) based on the observed associations (positive or negative) between the biomarkers described herein and the in vitro functionality of engineered cells, e.g., CAR T cells, derived from such donor cells. In one embodiment, a method of depletion or enrichment is provided including the step of providing donor cells from a donor cell population, wherein the donor cells comprise cells that express one or more of the following cell surface biomarkers: HLA-DR, TIGIT, CD16, CD56, CD27 CCR7, CD45RA, and any combination thereof. Table 1-10 provide different biomarker profiles for such donor cells. In another embodiment, the method further comprises depleting or removing cells from the donor cells that express one or more of the following cell surface biomarkers: HLA-DR, TIGIT, CD16, CD56, and any combination thereof. In other embodiment, the method further comprises enriching for or retaining cells from the donor cells that express one or more of the following cell surface biomarkers: CD27 CCR7, CD45RA, and any combination thereof.

According to the instant disclosure, the enrichment and/or depletion of cells from donor cells may be performed using flow cytometry approaches. The isolation of particles or cells (e.g., donor cells from a donor cell population and/or engineered cells from an engineered cell population) may be achieved by the addition of a sorting or collection function to a flow cytometer. Particles or cells that are spaced in a fluid stream may be detected as having one or more desired characteristics and subsequently isolated based on the detected characteristic(s) for each individual particle or cell. Particles or cells can be individually isolated from the stream by mechanical or electrical removal.

In one aspect, the instant disclosure provides methods and reagents for the removal of unwanted cells from a cell population (e.g., unwanted donor cells from a donor cell population and/or unwanted engineered cells from an engineered cell population) in a process of cell depletion. The unwanted cells can be depleted based on their expression one or more biomarkers that correlate to less in vitro functionality. Initially, cells from a cell population are exposed to one or more biomarker depletion reagents. In some embodiments, the biomarker depletion reagent(s) comprises an antibody targeting a biomarker expressed on the surface of donor cells (e.g., a cell surface protein, such as HLA-DR, TIGIT, CD16, CD56, and any combination thereof).

The anti-biomarker antibody, or any other antibody can be conjugated, for example, to biotin to facilitate further labeling and/or separation using a secondary antibody (e.g., an anti-biotin antibody). The secondary antibody can be conjugated either directly or indirectly with a magnetic depletion reagent such as magnetic depletion agent such as magnetic microbeads (nanoparticles that are generally, but not necessarily, about 50 nm in diameter) or any other surface, such as an agarose bead, an acoustic wave particle, a plastic welled plate, a glass welled plate, a ceramic welled plate, a column, a cell culture bag, or a membrane. When magnetic microbeads are used, the microbeads facilitate separation of the biomarker-positive cells from the biomarker-negative cells; when contacted with a magnetic column, the biomarker-positive cells can be retained on the column while unlabeled biomarker-negative cells pass through to a collection bag. Acoustic wave particles can facilitate separation of biomarker-positive from the biomarker-negative cells when exposed to an acoustic wave. While an anti-biotin antibody is provided in the context of the disclosed method, other biotin-binding partners such as streptavidin, avidin, and other proteins that recognize biotin can be employed in lieu of an anti-biotin antibody in all the methods provided herein.

In some embodiments, the anti-biomarker antibody specific for the biomarker is optionally conjugated to a fluorophore. In this embodiment, the step of sorting or selecting the donor cells that bind to the antibody (e.g., a monoclonal antibody) can be done by Fluorescence Activated Cell Sorting (FACS).

In some other embodiments, the anti-biomarker antibody specific for the biomarker is optionally conjugated to a magnetic particle. In this embodiment, the step of sorting or selecting the cells that bind to the antibody (e.g., a monoclonal antibody) can be done by Magnetic Activated Cell Sorting (MACS).

In some embodiments of the disclosed methods, sorting or separating biomarker-positive cells from biomarker-negative cells can be achieved using Magnetic-Activated Cell Sorting (MACS). Magnetic-activated cell sorting is a method for separation of various cell populations depending on their surface antigens (CD molecules) by using superparamagnetic nanoparticles and columns. MACS can be used to obtain a very pure donor cell population. Donor cells from a donor cell population in a single-cell suspension can be magnetically labeled with microbeads. The sample is applied to a column composed of a ferromagnetic material, which is covered with a coating not disruptive to cells, thus allowing fast and gentle separation of cells. The unlabeled cells pass through the column while the magnetically labeled cells are retained within the column. The flow-through can be collected as the unlabeled cell fraction. After a washing step, the column is removed from the separator, and the magnetically labeled cells are eluted from the column.

In some embodiments of the disclosed methods, sorting or separating biomarker-positive cells from biomarker-negative cells can be achieved using acoustic wave separation in lieu of magnetic-based separation methods. While not wishing to be bound by theory, it is understood that acoustic wave separation relies on a three-dimensional standing wave to separate components of a mixture. In the context of the disclosed methods, an anti-biomarker antibody, such as an antibody with specificity to one or more biomarkers, e.g., HLA-DR, TIGIT, CD16, or CD56, can be conjugated to a surface, such as an acoustic wave particle. An acoustic wave particle can be a bead. In an embodiment, donor cells from a donor cell population are exposed to acoustic wave particles bearing one or more anti-biomarker antibodies associating the acoustic wave particle with any cells expressing the target of interest. The cells are then placed in an acoustic chamber and exposed to an acoustic wave. Given the different properties of the bead-associated cells and cells that were not labeled with the antibody-bead particles, the acoustic wave separates the labeled and unlabeled cells, which can be collected while labeled cells (e.g., biomarker-positive cells) can be divert away from the unlabeled cells.

In some embodiments, the cells are analyzed for other surface markers indicative of different cell types in a population of cells, for example, effector cells, effector memory cells, central memory cells, stem central memory cells, etc. based on well-accepted specific surface markers for each cell type. In a further aspect, provided herein are methods of detecting surface markers indicative of other attributes of donor cells from a donor cell population. In some embodiments, the donor cells are analyzed for additional surface markers, the levels of which may indicate the potency or functionality of the cells. The analysis can be qualitative or quantitative.

### Cytokine Profiling

In one aspect, the present disclosure provides methods and reagents to analyze cytokine secretion profiles of cells from cell populations (e.g., donor cells from a donor cell population and/or engineered cells from an engineered cell population). Measuring secreted proteins, such as cytokines, from the cells of the cell population can provide information on cell attributes that may contribute to and/or correlate to an effect on the in vitro functionality of modified or engineered cells, e.g., CAR T cells, derived from donor cells. Flow cytometry methods, as described herein, can be used to generate secreted protein profiles, e.g., cytokine secretion profiles, on a single cell level. For example, a standard “cytokine secretion assay” (CSA) can be used to provide a cytokine secretion profile where a secreted cytokine of interest is characterized through the use of cell surface labeling agents specific to a component of cells from a cell population. For example, a bifunctional antibody, e.g., bispecific for a cell surface marker and a cytokine, may be used to stain cells and “catch” secreted cytokines at the cell surface. The cells may then be stained with a fluorescently labeled antibody specific to the caught cytokine. The stained cells are subsequently analyzed using fluorescent-activated cell sorting (FACS) to detect the cytokines on a single cell level.

Single-cell functional phenotypes, such as secretome profiles, e.g., cytokine secretion profiles, may be generated using discrete fluidic chambers and multiplexed enzyme-linked immunosorbent assay (ELISA) cytokine capture reagents, e.g., IsoCode chip using the IsoLight System (Isoplexis). In one aspect, the present disclosure provides methods for generating cytokine secretion profiles from cells of a cell population (e.g., donor cells of a donor cell population and/or engineered cells of an engineered cell population). In one embodiment, the method comprises partitioning of cells from a cell population onto an array of separate chambers (e.g., wells, troughs, cavities, depressions, channels, etc.), wherein a chamber (from the array of chambers) comprises one or more primary cytokine binding agents, e.g., primary antibodies specific for different cytokines, for capture of cytokines secreted from cells, e.g., donor cells from a donor cell population and/or engineered cells from an engineered cell population. In other embodiments, the chamber(s) is/are configured to hold a single cell(s). Optionally, the method comprises contacting the cells with an activation agent prior to partitioning. In one embodiment, the one or more primary cytokine binding agents are disposed or positioned on a surface (e.g., a planar surface) of the chamber and are configured to capture cytokines secreted from a cell that is within the chamber. In another embodiment, the method comprises subjecting a partition, e.g., a chamber, comprising a cell to conditions that allow one or more primary cytokine binding agents to bind to cytokines secreted from the cell. In other embodiments, the method further comprising removing the cells from the chamber while retaining one or more secreted cytokines bound to the one or more primary cytokine binding agents disposed on the surface. The method may further comprise contacting the bound secreted cytokines with one or more secondary cytokine binding agents, e.g., secondary antibodies specific for the one or more bound secreted cytokines, under conditions sufficient to allow the one or more secondary cytokine binding agents to bind the bound secreted cytokine. In other embodiments, the one or more secondary cytokine binding agents comprise one or more labels (as further described herein in the context of labeling agents). Such labels may be used to detect binding events, e.g., binding of a fluorescently labelled secondary cytokine binding agent to a bound secreted cytokine in the chamber, thereby providing a cytokine secretion profile for cells from a cell population. Additional details for methods and compositions are provided in Paczkowski et al. WO/2016/090148, which is incorporated herein by reference in its entirety. Cytokine secretion profiles may be expressed as a polyfunctionality index (PSI).

In another embodiment, the engineered immune cell population described herein is characterized by improved in vitro functionality, wherein the improved in vitro functionality comprises decreased cytokine secretion. Cytokine secretion can be measured by polyfunctional index score (PSI) as described in Example 2. In one embodiment, the engineered immune cell population exhibits decreased cytokine secretion as shown by a decreased PSI. In other embodiments, the decreased PSI is relative to a PSI observed for an engineered immune cell population derived from a non-healthy donor, e.g., a subject having a disease, such as cancer. In some embodiments, the PSI can be measured for the engineered immune cell population or a subpopulation thereof. In one embodiment, the subpopulation is a CD4+ subpopulation or a CD8+ subpopulation from the engineered immune cell population. In one embodiment, the PSI of the CD4+ subpopulation is between about 197 and about 549.

In one embodiment, the PSI of the CD8+ subpopulation is between about 155 and about 383.

Secreted cytokines may also be captured and detected at a single cell level using next generation sequencing techniques. Briefly, cells of a cell population (e.g., donor cells of a donor cell population and/or engineered cells of an engineered cell population) are contacted with a labeling agent that is conjugated to a reporter nucleic acid molecule to provide a labelled cell (e.g., labelled donor cell and/or labelled engineered cell). In one embodiment, the labeling agent is capable of labeling (e.g., binding to) a secreted cytokine. The reporter nucleic acid molecule comprises a reporter sequence that corresponds to the labeling agent (e.g., identifies the labeling agent and/or the cellular component that the labeling agent specifically labels, such as an antibody that specifically labels or binds a cytokine). In one embodiment, the labelled cell comprises a complex coupled to a surface of the cell. In another embodiment, the complex comprises a capture agent, a secreted cytokine, and the labeling agent. In one embodiment, the capture agent is configured to bind to both a cell surface protein of the cell and the secreted cytokine. In an additional embodiment, the labelled cell comprises (i) the capture agent bound to a cell surface protein and the secreted cytokine, and (ii) the labeling agent bound to the secreted cytokine. In another embodiment, the cells are contacted with the capture agent prior to contacting with the labeling agent, and optionally the cells are contacted with an activation agent, e.g., an immune cell activation agent, prior to contacting with the capture agent and/or labeling agent. Additional details for methods and compositions are provided in McDermott et al. WO/2021/072314, which is incorporated herein by reference in its entirety.

The cell populations described herein (e.g., donor cell populations and/or engineered cell populations) can be analyzed for secretion of various cytokines including, without limitation, one or more of granzyme B, IFN-gamma, MIP-1alpha, perforin, TNF-alpha, TNF-beta, GM-CSF, IL-2, IL-5, IL-7, IL-8, IL-9, IL-12, IL-5, IL-21, CCL11, IP-10, MIP-1beta, RANTES, IL-2, IL-10, IL-13, IL-22, TGF-beta1, sCD137, sCD40L, IL-1beta, IL-6, IL-17A, IL-17F, MCP-1, MCP-4, and any combination thereof.

### Determination of In Vitro Functionality of CAR T Cells

In this further aspect, the instant disclosure provides a method for analyzing and/or determining in vitro functionality (including one or more of potency, i.e., cytotoxicity, cell fitness, and/or polyfunctionality, i.e., cytokine secretion profiling) of an engineered cell derived from a donor cell of a donor cell population. In some embodiments, the donor cell is an immune cell. In other embodiments, the engineered cell is an engineered immune cell, for example, a CAR T cell. Currently, there are different methods for evaluating the in vitro functionality of CAR T products. Upon exposing/binding to target cells, CAR T cells exert cytotoxicity partly through secretion of one or more effector cytokines. Effective cytokine induction can be used as an indication for potency or polyfunctionality of CAR T cells. Secreted cytokine can be measured by an immune assay such as ELISA. Cytokine induction of CAR T cells can also be assessed by intracellular staining after fixation of cells.

Potency, as used herein, can refer to the ability of one or more immune cells, such as CAR T cells derived from donor cells of a donor cell population, to kill a target cell, such as an antigen positive tumor cell.

Polyfunctionality, as used herein, can refer to the ability of one or more immune cells, such as CAR T cells derived from donor cells of a donor cell population, to secrete more than one effector cytokine or molecule upon target or antigen activation. In some embodiments, polyfunctional CAR T cells secrete two or more effector cytokines, or three or more effector cytokines, upon target or antigen activation.

The instant disclosure provides data demonstrating correlations between cell surface expression of certain biomarkers (e.g., donor cell surface expression and/or engineered cell surface expression) and in vitro functionality of engineered cells derived from such donor cells. As described herein, the markers include negative association markers HLA-DR, TIGIT, CD16, and CD56, and positive association markers CD27, CCR7, and CD45RA. HLA-DR is typically considered an activation marker and Saraiva et al. reported that its expression in T cells positively associated with chemotherapy response in breast cancer patients (HLA-DR in Cytotoxic T Lymphocytes Predicts Breast Cancer Patients Response to Neoadjuvant Chemotherapy. Front Immunol. 2018 Nov. 13; 9:2605. Doi: 10.3389/fimmu.2018.02605).

In one aspect, the instant disclosure provides a method of detecting (i) a level of surface expression of a negative association biomarker, e.g., HLA-DR, TIGIT, CD16, CD56, and any combination thereof, and/or (ii) a level of surface expression of a positive association marker, e.g., CD27, CCR7, CD45RA, and any combination thereof, in a cell population as described herein (e.g., see Tables 1-10). In one embodiment, the (i) level of surface expression of a negative association biomarker and/or (ii) the level of surface expression of a positive association biomarker can be used as a proxy or indicator for in vitro functionality of the cell population. The cell population may be a donor cell population (prior to any genetic modification) or an engineered cell population, such as a genetically modified cell population, e.g., a CAR T cell population, that has been derived from the donor cell population.

In one embodiment, the detecting step is performed after exposing the cell population to an activation agent. In vitro manipulation, e.g., selection, culturing and expansion, of immune cell populations often includes the use of reagents that activate or stimulate T cells. Such activation or stimulation is an important part of the process of selecting and expanding single cell clones. In one additional aspect, the instant disclosure provides in vitro methods for the manipulation of donor cell populations that comprise contacting donor cells of a donor cell population and/or engineered cells, e.g., engineered immune cells (such as CART cells), derived from donor cells of a donor cell population with one or more activation agents. Suitable activation agents include, without limitation, a CD3 activation agent and/or a CD28 activation agent. In one embodiment, the activation agent comprises one or more agents coupled to a support (such as a bead or a matrix, such as a polymeric matrix). In another embodiment, the CD3 activation agent comprises an anti-CD3 antibody and/or the CD28 activation agent comprises an anti-CD28 antibody. In one other embodiment, the activation agent comprises an IL2R (including IL2Rα, IL2β, and IL2Rγ) ligand, such as IL-2 or an IL-2 mimetic.

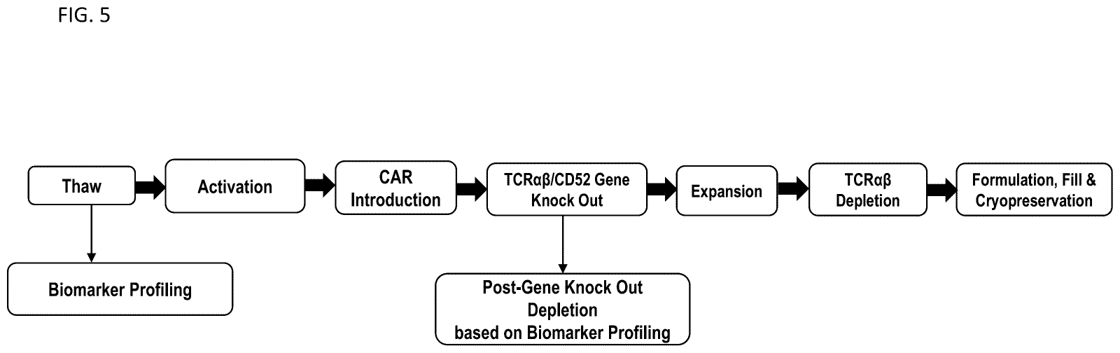

In another aspect of the instant disclosure, cell populations can be contacted with or exposed to an activation agent at one or more time points during the process of manufacturing engineered cells from donor cells, as described herein. FIG. 5 depicts an exemplary workflow for the manufacturing of engineered immune cells from donor cells from donor cell populations as described herein.

In another embodiment, the detecting step may be performed after contacting or exposing engineered immune cells that are specific to a target molecule to the target molecule. For example, CAR T cells expressing a CAR specific for an antigen may be exposed to the antigen or to target cells that express the antigen. Antigen stimulation (or antigen activation) of CAR T cells can be achieved by, for example, binding to antigens, binding to target cells, e.g., target tumor cells expressing the antigen, or by co-culturing with target cells, e.g., target tumor cells expressing the antigen. In some embodiments, one or more surface biomarkers (positive or negative association) of CAR T cells as described herein is measured between about 4 hours and about 10 hours after antigen activation. In other embodiments, the one or more surface biomarkers of CART cells are measured about 4 hours, about 5 hours, about 6 hours, about 7 hours, about 8 hours, about 9 hours, or about 10 hours after antigen activation. In some embodiments, the one or more surface biomarkers of CAR T cells are measured about 4 hours, about 5 hours, about 6 hours, about 7 hours, about 8 hours, about 9 hours, or about 10 hours after co-culturing with target cells. In some embodiments, the one or more surface biomarkers of CART cells are measured about 6 hours after co-culturing with target cells. In some embodiments, the CAR T cells are autologous CAR T cells. In some embodiments, the CAR T cells are allogeneic CAR T cells.

In some embodiments, the CAR T cells are autologous CAR T cells. In some embodiments, the CAR T cells are allogeneic CAR T cells. In some embodiments, the CAR T cells are TCR− allogeneic CAR T cells. In contrast to TCR+ CAR T cells, in the absence of a functional TCR, the activation of the engineered T cells relies on the interaction of the CAR with the antigen.

The instant disclosure also provides an overall process for screening, characterizing and analyzing cell populations, such as donor cells from a donor cell population and/or engineered cell populations, e.g., CAR T cells, derived from such donor cells. Accordingly, in one aspect, the instant disclosure provides a method for analyzing a population of donor cells, such as donor immune cells, or engineered donor cells, such as engineered donor immune cells, for example, allogeneic CAR T cells, comprising steps of measuring or determining a percentage or amount of T cells that express one or more biomarkers in the population of cells, and determining in vitro functionality of the population of cells.

In some embodiments, the method further comprises the step of measuring a percentage or amount of CAR+ T cells. In some embodiments, the percentage or amount of CAR+ T cells can be determined by using a reagent, for example, an anti-id antibody or an antigen. The antigen can be soluble or immobilized on a solid surface. The reagent can be directly labeled for detection or bound by a secondary labeled reagent for detection. In some embodiments, the method further comprises the step of measuring or detecting CD52+ cells. In some embodiments, the CAR T cells are manufactured in a GMP manufacturing process. In some embodiments, the population of engineered immune cells are TCR− allogeneic CAR T cells manufactured in a GMP manufacturing process. In some embodiments, the population of engineered immune cells are GMP allogeneic CAR T drug substance or drug product.

In certain embodiments, the CAR T cells are specific for EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In certain embodiments, the CAR T cells are EGFRvIII CAR T cells, CD19 CAR T cells, CD20 CAR T cells, CD33 CAR T cells, ROR1 CAR T cells, CD70 CAR T cells, FLT3 CAR T cells, BCMA CAR T cells, or DLL3 CAR T cells. In certain embodiments, the CAR T cells are BCMA CAR T cells. In certain embodiments, the BCMA CAR T cells comprise the BCMA CAR comprising the sequence set forth in SEQ ID NO: 1 or SEQ ID NO:2.

(SEQ ID NO: 1)
E V Q L L E S G G G L V Q P G G S L R L S C A A S
G F T F S S Y A M N W V R Q A P G K G L E W V S A
I S D S G G S T Y Y A D S V K G R F T I S R D N S
K N T L Y L Q M N S L R A E D T A V Y Y C A R Y W
P M D I W G Q G T L V T V S S G G G G S G G G G S
G G G G S E I V L T Q S P G T L S L S P G E R A T
L S C R A S Q S V S S S Y L A W Y Q Q K P G Q A P
R L L M Y D A S I R A T G I P D R F S G S G S G T
D F T L T I S R L E P E D F A V Y Y C Q Q Y G S W
P L T F G Q G T K V E I K G S G G G G S C P Y S N
P S L C S G G G G S C P Y S N P S L C S G G G G S
T T T P A P R P P T P A P T I A S Q P L S L R P E
A C R P A A G G A V H T R G L D F A C D I Y I W A
P L A G T C G V L L L S L V I T L Y C K R G R K K
L L Y I F K Q P F M R P V Q T T Q E E D G C S C R
F P E E E E G G C E L R V K F S R S A D A P A Y Q
Q G Q N Q L Y N E L N L G R R E E Y D V L D K R R
G R D P E M G G K P R R K N P Q E G L Y N E L Q K
D K M A E A Y S E I G M K G E R R R G K G H D G L
Y Q G L S T A T K D T Y D A L H M Q A L P P R
(SEQ ID NO: 2)
E V Q L L E S G G G L V Q P G G S L R L S C A A S
G F T F S S Y P M S W V R Q A P G K G L E W V S A
I G G S G G S L P Y A D S V K G R F T I S R D N S
K N T L Y L Q M N S L R A E D T A V Y Y C A R Y W
P M D I W G Q G T L V T V S S G G G G S G G G G S
G G G G S E I V L T Q S P G T L S L S P G E R A T
L S C R A S Q S V S S S Y L A W Y Q Q K P G Q A P
R L L M Y D A S I R A T G I P D R F S G S G S G T
D F T L T I S R L E P E D F A V Y Y C Q Q Y Q S W
P L T F G Q G T K V E I K G S G G G G S C P Y S N
P S L C S G G G G S C P Y S N P S L C S G G G G S
T T T P A P R P P T P A P T I A S Q P L S L R P E
A C R P A A G G A V H T R G L D F A C D I Y I W A
P L A G T C G V L L L S L V I T L Y C K R G R K K
L L Y I F K Q P F M R P V Q T T Q E E D G C S C R
F P E E E E G G C E L R V K F S R S A D A P A Y Q
Q G Q N Q L Y N E L N L G R R E E Y D V L D K R R
G R D P E M G G K P R R K N P Q E G L Y N E L Q K
D K M A E A Y S E I G M K G E R R R G K G H D G L
Y Q G L S T A T K D T Y D A L H M Q A L P P R

### 1. Immune Cells

Donor cells from a donor cell population or engineered cells derived from the donor cells that are suitable for use with the methods and/or reagents described herein include immune cells.

Prior to the in vitro manipulation or genetic modification (e.g., as described herein), donor cells for use in methods described herein (e.g., immune cells) can be obtained from a subject. Donor cells can be obtained from a number of non-limiting sources, including peripheral blood mononuclear cells, bone marrow, lymph node tissue, cord blood, thymus tissue, stem cell- or iPSC-derived immune cells, tissue from a site of infection, ascites, pleural effusion, spleen tissue, and tumors. In some embodiments, any number of T cell lines available and known to those skilled in the art, can be used. In some embodiments, donor cells can be derived from a healthy donor, from a patient diagnosed with cancer or from a patient diagnosed with an infection. In some embodiments, donor cells can be part of a mixed population of cells which present different phenotypic characteristics.

In some embodiments, immune cells are autologous immune cells obtained from a subject who will ultimately receive the engineered immune cells. In some embodiments, immune cells are allogeneic immune cells obtained from a donor, who is a different individual from the subject who will receive the engineered immune cells.

In some embodiments, immune cells comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. In certain some embodiments, T cells can be obtained from a volume of blood collected from the subject using any number of techniques known to the skilled person, such as FICOLL™ separation.

Donor cells can be obtained from the circulating blood of an individual by apheresis. The apheresis product typically contains lymphocytes, including T cells, monocytes, granulocytes, B cells, other nucleated white blood cells, red blood cells, and platelets. In certain some embodiments, the cells collected by apheresis can be washed to remove the plasma fraction, and placed in an appropriate buffer or media for subsequent processing.

Donor PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In certain embodiments, T cells are isolated from PBMCs by lysing the red blood cells and depleting the monocytes, for example, using centrifugation through a PERCOLL™ gradient. A specific subpopulation of T cells, such as CCR7+, CD95+, CD122, CD27+, CD69+, CD127+, CD28+, CD3+, CD4+, CD8+, CD25+, CD62L+, CD45RA+, and CD45RO+ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

In one embodiment, to deplete unwanted cells that express one or more cell surface biomarkers, e.g., HLA-DR, TIGIT, CD16, CD56, and any combination thereof, one or more antibodies directed to such one or more cell surface biomarkers present on the unwanted cells may be used to enrich for cells that do not express the cell surface biomarkers.

In some embodiments, a population of donor cells, e.g., immune cells such as T cells, is enriched for CD4+ cells.

In some embodiments, a population of donor cells, e.g., immune cells such as T cells, is enriched for CD8+ cells.

In some embodiments, CD8+ cells are further sorted into naive, central memory, and effector cells by identifying cell surface antigens that are associated with each of these types of cells. In some embodiments the expression of phenotypic markers for naïve T cells include CD45RA+, CD95−, IL2Rβ−, CCR7+, and CD62L+. In some embodiments the expression of phenotypic markers for stem cell memory T cells include CD45RA+, CD95+, IL2Rβ+, CCR7+, and CD62L+. In some embodiments the expression of phenotypic markers for central memory T cells include CD45RO+, CD95+, IL2Rβ+, CCR7+, and CD62L+. In some embodiments the expression of phenotypic markers for effector memory T cells include CD45RO+, CD95+, IL2Rβ+, CCR7−, and CD62L−. In some embodiments the expression of phenotypic markers for T effector cells include CD45RA+, CD95+, IL2Rβ+, CCR7−, and CD62L−. Thus, CD4+ and/or CD8+ T helper cells can be sorted into naive, stem cell memory, central memory, effector memory and T effector cells by identifying cell populations that have cell surface antigens.

It will be appreciated that donor PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum. In various embodiments a crypreservative media can comprise, for example, CryoStor® CS2, CS5, or CS10 or other medium comprising DMSO, or a medium that does not comprise DMSO.

### 2. Engineered Immune Cells

Provided herein are engineered immune cells expressing the CARs of the disclosure (e.g., CAR-T cells) that have been derived from donor cells as described herein. In one embodiment, the engineered immune cells are (i) derived from donor cells of a donor cell population having a biomarker profile as described herein and (ii) characterized by improved in vitro functionality as compared to an engineered immune cell derived from donor cells that lack the biomarker profile.

In some embodiments, an engineered immune cell comprises a population of CARs, each CAR comprising extracellular antigen-binding domains. In some embodiments, an engineered immune cell comprises a population of CARs, each CAR comprising different extracellular antigen-binding domains. In some embodiments, an immune cell comprises a population of CARs, each CAR comprising the same extracellular antigen-binding domains.

The engineered immune cells can be allogeneic or autologous.

In some embodiments, the engineered immune cell is a T cell (e.g., inflammatory T-lymphocyte cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell.

In one embodiment, the engineered immune cell can be derived from a donor cell with a biomarker profile as described herein. In some embodiments, the engineered immune cell can be derived from the group consisting of CD4+ T-lymphocytes and CD8+ T-lymphocytes. In some exemplary embodiments, the engineered immune cell is a T cell. In some exemplary embodiments, the engineered immune cell is an alpha beta T cell. In some exemplary embodiments, the engineered immune cell is a gamma delta T cell. In some exemplary embodiments, the engineered immune cell is a macrophage.

In some embodiments, the engineered immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells (iPSC), totipotent stem cells or hematopoietic stem cells. Stem cells can be CD34+ or CD34−.

In some embodiments, the donor cells are obtained or prepared from peripheral blood. In some embodiments, the donor cells are obtained or prepared from peripheral blood mononuclear cells (PBMCs). In some embodiments, the donor cells are obtained or prepared from bone marrow. In some embodiments, the donor cells are obtained or prepared from umbilical cord blood. In some embodiments, the donor cells are human cells. In some embodiments, the transfected or transduced by the nucleic acid vector using a method selected from the group consisting of electroporation, sonoporation, biolistics (e.g., Gene Gun), transfection, lipid transfection, polymer transfection, nanoparticles, viral transduction or viral transfection (e.g., retrovirus, lentivirus, AAV) or polyplexes. In some embodiments the donor cell is a T cell that has been re-programmed from a non-T cell. In some embodiments the donor cell is a T cell that has been re-programmed from a T cell.

### Binding Agents (Including Antibodies and Fragments Thereof)

In embodiments, the disclosed methods for biomarker identification or detection comprise the use of an antibody or antigen binding agent (e.g., comprising an antigen binding domain or comprising an antibody or fragment thereof). As discussed below, in various embodiments engineered immune cells derived from donor cells of a donor cell population can also comprise a binding agent.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHI, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence.

Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the instant disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art.

Moreover, the term “antibody” as used herein, can refer to any of the art-known or developed constructs or formats for utilizing antibody structural and functional features in alternative presentation. For example, in some embodiments, an antibody utilized in the methods of the instant disclosure is in a format selected from, but not limited to, intact IgA, IgG, IgE or IgM antibodies; bi- or multi-specific antibodies (e.g., Zybodies®, etc.); antibody fragments such as Fab fragments, Fab fragments, F(ab)2 fragments, Fd fragments, and isolated CDRs or sets thereof; single chain variable fragments (scFVs); polypeptide-Fc fusions; single domain antibodies (e.g., shark single domain antibodies such as IgNAR or fragments thereof); camelid antibodies (also referred to herein as nanobodies or VHHs); shark antibodies, masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

As used herein, the term “antibody agent” generally refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc. as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

An antibody or antibody agent used in performing the methods of the instant disclosure can be single chained or double chained. In some embodiments, the antibody or antigen binding molecule is single chained. In certain embodiments, the antigen binding molecule is selected from the group consisting of an scFv, a Fab, a Fab′, a Fv, a F(ab′)₂, a dAb, and any combination thereof.

Antibodies and antibody agents include antibody fragments. An antibody fragment comprises a portion of an intact antibody, such as the antigen binding or variable region of the intact antibody. Antibody fragments include, but are not limited to, Fab, Fab′, Fab′-SH, F(ab′)₂, Fv, diabody, linear antibodies, multispecific formed from antibody fragments antibodies and scFv fragments, and other fragments. Antibodies also include, but are not limited to, polyclonal monoclonal, chimeric dAb (domain antibody), single chain, Fab, Fa, F(ab′)₂ fragments, and scFvs. An antibody can be a whole antibody, or immunoglobulin, or an antibody fragment. Antibody fragments can be made by various techniques, including but not limited to proteolytic digestion of an intact antibody as well as production by recombinant host cells (e.g., E. coli, Chinese Hamster Ovary (CHO) cells, or phage), as known in the art.

In some embodiments, an antibody or antibody agent can be a chimeric antibody (see, e.g., U.S. Pat. No. 4,816,567; and Morrison et al., Proc. Natl. Acad. Sci. USA, 81:6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci., 13:1619-1633 (2008); Riechmann et al., Nature, 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol, 28:489-498 (1991); Dall'Acqua et al., Methods, 36:43-60 (2005); Osbourn et al., Methods, 36:61-68 (2005); and Klimka et al., Br. J. Cancer, 83:252-260 (2000)). A humanized antibody is a chimeric antibody comprising amino acid residues from non-human hypervariable regions and amino acid residues from human FRs. In certain embodiments, a humanized antibody will comprise substantially all of at least one, and typically two, variable domains, in which all or substantially all of the hypervariable regions (e.g., CDRs) correspond to those of a non-human antibody, and all or substantially all of the Framework Regions (FRs) correspond to those of a human antibody. A humanized antibody optionally can comprise at least a portion of an antibody constant region derived from a human antibody.

In some embodiments, an antibody or antibody agent provided herein is a human antibody. Human antibodies can be produced using various techniques known in the art (See, e.g., van Dijk and van de Winkel, Curr. Opin. Pharmacol, 5: 368-74 (2001); and Lonberg, Curr. Opin. Immunol, 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared using methods well known in the art.

### Chimeric Antigen Receptors

As used herein, chimeric antigen receptors (CARs) are proteins that specifically recognize target antigens (e.g., target antigens on cancer cells). When bound to the target antigen, the CAR can activate the immune cell to attack and destroy the cell bearing that antigen (e.g., the cancer cell). CARs can also incorporate costimulatory or signaling domains to increase their potency. See Krause et al., J. Exp. Med., Volume 188, No. 4, 1998 (619-626); Finney et al., Journal of Immunology, 1998, 161: 2791-2797, Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016); U.S. Pat. Nos. 7,741,465, and 6,319,494.

Chimeric antigen receptors described herein comprise an extracellular domain, a transmembrane domain, and an intracellular domain, wherein the extracellular domain comprises an antigen binding domain that specifically binds to the target.

In some embodiments, antigen-specific CARs further comprise a safety switch and/or one or more monoclonal antibody specific-epitope.

i. Antigen Binding Domains

As discussed above, CARs described herein comprise an antigen binding domain. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease.

In some embodiments, the antigen binding domain comprises a variable heavy chain, variable light chain, and/or one or more CDRs described herein. In some embodiments, the antigen binding domain is a single chain variable fragment (scFv), comprising light chain CDRs CDR1, CDR2 and CDR3, and heavy chain CDRs CDR1, CDR2 and CDR3.

An antigen binding domain is said to be “selective” when it binds to one target more tightly or with higher affinity than it binds to a second target.

The antigen binding domain of the CAR selectively targets a cancer antigen. In some embodiments, the cancer antigen is selected from EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In some embodiments, the CAR comprises an antigen binding domain that targets EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34.

In some embodiments, the cancer antigen is selected from the group consisting of carbonic anhydrase IX (CAIX), carcinoembryonic antigen (CEA), CDS, CD7, CDIO, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AchR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis A (CA19.9), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), and Wilms tumor protein (WT-1).

Variants of the antigen binding domains (e.g., variants of the CDRs, VH and/or VL) are also within the scope of the disclosure, e.g., variable light and/or variable heavy chains that each have at least 70-80%, 80-85%, 85-90%, 90-95%, 95-97%, 97-99%, or above 99% identity to the amino acid sequences of antigen binding domain sequences. In some instances, such molecules include at least one heavy chain and one light chain, whereas in other instances the variant forms contain two variable light chains and two variable heavy chains (or subparts thereof). A skilled artisan will be able to determine suitable variants of the antigen binding domains as set forth herein using well-known techniques. In certain embodiments, one skilled in the art can identify suitable areas of the molecule that can be changed without destroying activity by targeting regions not believed to be important for activity.

In certain some embodiments, the polypeptide structure of the antigen binding domains is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding domain comprises or consists of avimers.

In some embodiments, an antigen binding domain is a scFv.

In some embodiments, an antigen-selective CAR comprises a leader or signal peptide.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In some embodiments, a CAR-immune cell (e.g., CAR-T cell) which can form a component of an engineered immune cell population (derived from donor cells of a donor cell population as described herein) generated by practicing the methods of the instant disclosure comprises a polynucleotide encoding a safety switch polypeptide, such as for example RQR8. See, e.g., WO2013153391A, which is hereby incorporated by reference in its entirety. In a CAR-immune cell (e.g., a CAR-T cell) comprising the polynucleotide, the safety switch polypeptide can be expressed at the surface of a CAR-immune cell (e.g., CAR-T cell).

ii. Hinge Domain

The extracellular domain of the CARs of the disclosure can comprise a “hinge” domain (or hinge region). The term generally refers to any polypeptide that functions to link the transmembrane domain in a CAR to the extracellular antigen binding domain in a CAR. In particular, hinge domains can be used to provide more flexibility and accessibility for the extracellular antigen binding domain.

A hinge domain can comprise up to 300 amino acids—in some embodiments 10 to 100 amino acids or in some embodiments 25 to 50 amino acids. The hinge domain can be derived from all or part of naturally occurring molecules, such as from all or part of the extracellular region of CD8, CD4, CD28, 4-1BB, or IgG (in particular, the hinge region of an IgG; it will be appreciated that the hinge region can contain some or all of a member of the immunoglobulin family such as IgG1, IgG2, IgG3, IgG4, IgA, IgD, IgE, IgM, or fragment thereof), or from all or part of an antibody heavy-chain constant region. Alternatively, the hinge domain can be a synthetic sequence that corresponds to a naturally occurring hinge sequence, or can be an entirely synthetic hinge sequence. In some embodiments said hinge domain is a part of human CD8a chain (e.g., NP 001139345.1). In other embodiments, said hinge and transmembrane domains comprise a part of human CD8a chain. In some embodiments, the hinge domain of CARs described herein comprises a subsequence of CD8a, an IgG1, IgG4, PD-1 or an FcγRIIIa, in particular the hinge region of any of an CD8a, an IgG1, IgG4, PD-1 or an FcγRIIIa. In some embodiments, the hinge domain comprises a human CD8a hinge, a human IgG1 hinge, a human IgG4, a human PD-1 or a human FcγRIIIa hinge. In some embodiments the CARs disclosed herein comprise a scFv, CD8a human hinge and transmembrane domains, the CD3t signaling domain, and 4-1BB signaling domain.

iii. Transmembrane Domain

The CARs of the disclosure are designed with a transmembrane domain that is fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. In some embodiments, short linkers can form linkages between any or some of the extracellular, transmembrane, and intracellular domains of the CAR.

Suitable transmembrane domains for a CAR disclosed herein have the ability to (a) be expressed at the surface an immune cell such as, for example without limitation, a lymphocyte cell, such as a T helper (Th) cell, cytotoxic T (Ta) cell, T regulatory (Treg) cell, or Natural killer (NK) cells, and/or (b) interact with the extracellular antigen binding domain and intracellular signaling domain for directing the cellular response of an immune cell against a target cell.

The transmembrane domain can be derived either from a natural or from a synthetic source. Where the source is natural, the domain can be derived from any membrane-bound or transmembrane protein.

Transmembrane regions of particular use in this disclosure can be derived from (comprise, or correspond to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

As non-limiting examples, the transmembrane region can be derived from, or be a portion of a T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1).

In some embodiments, the transmembrane domain in the CAR of the disclosure is a CD8α transmembrane domain.

In some embodiments, the transmembrane domain in the CAR of the disclosure is a CD28 transmembrane domain.

iv. Intracellular Domain

The intracellular (cytoplasmic) domain of the CARs of the disclosure can provide activation of at least one of the normal effector functions of the immune cell comprising the CAR. Effector function of a T cell, for example, can refer to cytolytic activity or helper activity, including the secretion of cytokines.

In some embodiments, an activating intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

It will be appreciated that suitable (e.g., activating) intracellular domains include, but are not limited to signaling domains derived from (or corresponding to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

The intracellular domains of the CARs of the disclosure can incorporate, in addition to the activating domains described above, co-stimulatory signaling domains (interchangeably referred to herein as costimulatory molecules) to increase their potency. Co-stimulatory domains can provide a signal in addition to the primary signal provided by an activating molecule as described herein.

It will be appreciated that suitable co-stimulatory domains within the scope of the disclosure can be derived from (or correspond to) for example, CD28, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 1a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNFR, integrin, signaling lymphocytic activation molecule, BTLA, Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the disclosure.

In some embodiments, the intracellular/cytoplasmic domain of the CAR can be designed to comprise the 4-1BB/CD137 domain by itself or combined with any other desired intracellular domain(s) useful in the context of the CAR of the disclosure. The complete native amino acid sequence of 4-1BB/CD137 is described in NCBI Reference Sequence: NP_001552.2. The complete native 4-1BB/CD137 nucleic acid sequence is described in NCBI Reference Sequence: NM_001561.5.

In some embodiments, the intracellular/cytoplasmic domain of the CAR can be designed to comprise the CD28 domain by itself or combined with any other desired intracellular domain(s) useful in the context of the CAR of the disclosure. The complete native amino acid sequence of CD28 is described in NCBI Reference Sequence: NP_006130.1. The complete native CD28 nucleic acid sequence is described in NCBI Reference Sequence: NM_006139.1.

In some embodiments, the intracellular/cytoplasmic domain of the CAR can be designed to comprise the CD3 zeta domain by itself or combined with any other desired intracellular domain(s) useful in the context of the CAR of the disclosure.

For example, the intracellular domain of the CAR can comprise a CD3 zeta chain portion and a portion of a costimulatory signaling molecule. The intracellular signaling sequences within the intracellular signaling portion of the CAR of the disclosure can be linked to each other in a random or specified order. In some embodiments, the intracellular domain is designed to comprise the activating domain of CD3 zeta and a signaling domain of CD28. In some embodiments, the intracellular domain is designed to comprise the activating domain of CD3 zeta and a signaling domain of 4-1BB.

In some embodiments the intracellular signaling domain of the CAR of the disclosure comprises a domain of a co-stimulatory molecule. In some embodiments, the intracellular signaling domain of a CAR of the disclosure comprises a part of co-stimulatory molecule selected from the group consisting of fragment of 4-1BB (GenBank: AAA53133.) and CD28 (NP_006130.1).

### Engineered Immune Cells Comprising CARs

Also provided herein are engineered immune cells and populations of engineered immune cells expressing CAR (e.g., CAR-T cells or CAR+ cells), which are derived from donor cells having a biomarker profile that has been detected according to the methods described herein. Such engineered immune cells may be cells that have been depleted of cells expressing one or more unwanted biomarkers (e.g., HLA-DR, TIGIT, CD16, CD56, and any combination thereof) and/or endogenous TCR.

In some embodiments, an engineered immune cell comprises a CAR T cell, each CAR T cell comprising an extracellular antigen-binding domain and has reduced or eliminated expression of one or more wanted biomarkers (as described herein) and/or endogenous TCR. In some embodiments, a population of engineered immune cells comprises a population of CAR T cells, each CAR T cell comprising two or more different extracellular antigen-binding domain and has reduced or eliminated expression of endogenous TCR. In some embodiments, an immune cell comprises a population of CARs, each CAR T cell comprising the same extracellular antigen-binding domains and has reduced or eliminated expression of one or more wanted biomarkers (as described herein) and/or endogenous TCR.

The engineered immune cells can be allogeneic or autologous.

In some embodiments, an engineered immune cell or population of engineered immune cells is a T cell (e.g., inflammatory T-lymphocyte cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell, and expresses a CAR. In some embodiments, the T cell can be derived from the group consisting of CD4+ T lymphocytes, CD8+T lymphocytes or population comprising a combination of CD4+ and CD8+ T cells.

In some embodiments, an engineered immune cell or population of engineered immune cells that are generated using the disclosed methods can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, an engineered immune cell or a population of immune cells that are generated using the disclosed methods is obtained or prepared from peripheral blood, wherein the peripheral blood comprises donor cells from a donor cell population having a biomarker profile as described herein. In some embodiments, an engineered immune cell is obtained or prepared from peripheral blood mononuclear cells (PBMCs). In some embodiments, an engineered immune cell is obtained or prepared from bone marrow. In some embodiments, an engineered immune cell is obtained or prepared from umbilical cord blood. In some embodiments, the donor cell is a human cell. In some embodiments, the donor cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

In some embodiments, the engineered immune cells expressing at their cell surface membrane an antigen-specific CAR comprise a percentage of stem cell memory and central memory cells greater than 10%, 20%, 30%, 40%, 50%, 60%, 70%, 80%, 90%, or 100%.

In some embodiments, engineered immune cells expressing at their cell surface membrane an antigen-specific CAR comprise a percentage of stem cell memory and central memory cells of about 10% to about 60%, about 10% to about 50%, about 10% to about 40%, about 15% to about 50%, about 15% to about 40%, about 20% to about 60%, or about 20% to about 70%.

In some embodiments, engineered immune cells expressing at their cell surface membrane an antigen-specific CAR enriched in T_{CM} and/or T_{SCM} cells such that the engineered immune cells comprise at least about 60%, 65%, 70%, 75%, or 80% combined T_{CM} and T_{SCM} cells. In some embodiments, engineered immune cells expressing at their cell surface membrane an antigen-specific CAR are enriched in T_{CM} and/or T_{SCM} cells such that the engineered immune cells comprise at least about 70% combined T_{CM} and T_{SCM} cells. In some embodiments, engineered immune cells expressing at their cell surface membrane an antigen-specific CAR e enriched in T_{CM} and/or T_{SCM} cells such that the engineered immune cells comprise at least about 75% combined T_{CM} and/or T_{SCM} cells.

In some embodiments, an engineered immune cell is an inflammatory T-lymphocyte that expresses a CAR. In some embodiments, an engineered immune cell is a cytotoxic T-lymphocyte that expresses a CAR. In some embodiments, an engineered immune cell is a regulatory T-lymphocyte that expresses a CAR. In some embodiments, an engineered immune cell is a helper T-lymphocyte that expresses a CAR.

### Genetic Modification of CAR T Cells

In some embodiments, an engineered immune cell derived from donor cells having certain biomarker profiles according to the present disclosure can comprise one or more disrupted or inactivated genes. In some embodiments, a gene for a target antigen (e.g., EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70) can be knocked out to introduce a CAR targeting the same antigen (e.g., a EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70 CAR) to avoid induced CAR activation. As described herein, in some embodiments, an engineered immune cell according to the present disclosure comprises one disrupted or inactivated gene selected from the group consisting of MHC1 (β2M), MHC2 (CIITA), EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70, TCRα and TCRβ and/or expresses a CAR or a multi-chain CAR. In some embodiments, a cell comprises a multi-chain CAR. In some embodiments, the isolated cell comprises two disrupted or inactivated genes selected from the group consisting of: CD52 and TCRα, CDR52 and TCRβ, PD-1 and TCRα, PD-1 and TCRβ, MHC-1 and TCRα, MHC-1 and TCRβ, MHC2 and TCRα, MHC2 and TCRβ and/or expresses a CAR or a multi-chain CAR.

In some embodiments, an engineered immune cell derived from donor cells having certain biomarker profiles according to the present disclosure comprises one disrupted or inactivated gene selected from the group consisting of CD52, DLL3, GR, PD-1, CTLA-4, LAG3, TIM3, BTLA, BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two disrupted or inactivated genes selected from the group consisting of: CD52 and GR, CD52 and TCRα, CDR52 and TCRβ, DLL3 and CD52, DLL3 and TCRα, DLL3 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, TIM3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, including a multi-chain CAR, and/or a pTα transgene. In some embodiments the method comprises disrupting or inactivating one or more genes by introducing into the donor cells an endonuclease capable of selectively inactivating a gene by selective DNA cleavage. In some embodiments the endonuclease can be, for example, a zinc finger nuclease (ZFN), megaTAL nuclease, meganuclease, transcription activator-like effector nuclease (TALE-nuclease, or TALEN®), or CRISPR (e.g., Cas9 or Cas12) endonuclease.

In some embodiments, TCR is rendered not functional in the cells according to the disclosure by disrupting or inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used in for treating patients in need thereof against Host versus Graft (HvG) rejection and Graft versus Host Disease (GvHD); therefore in the scope of the present disclosure is a method of treating patients in need thereof against Host versus Graft (HvG) rejection and Graft versus Host Disease (GvHD) comprising treating said patient by administering to said patient an effective amount of modified cells comprising disrupted or inactivated TCRα and/or TCRβ genes.

The present disclosure provides methods of determining the purity of a population of engineered immune cells lacking or having reduced endogenous TCR expression. In some embodiments, the engineered immune cells comprise less than 5.0%, less than 4.0%, less than 3.0% TCR+ cells, less than 2.0% TCR+ cells, less than 1.0% TCR+ cells, less than 0.9% TCR+ cells, less than 0.8% TCR+ cells, less than 0.7% TCR+ cells, less than 0.6% TCR+ cells, less than 0.5% TCR+ cells, less than 0.4% TCR+ cells, less than 0.3% TCR+ cells, less than 0.2% TCR+ cells, or less than 0.1% TCR+ cells. Such a population can be a product of the disclosed methods.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, cyclophosphamide, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production.

In some embodiments, isolated cells or cell lines of the disclosure can comprise a pTα or a functional variant thereof. In some embodiments, an isolated cell or cell line can be further genetically modified by disrupting or inactivating the TCRα gene.

The disclosure also provides engineered immune cells (that are derived from donor cells having certain biomarker profiles) that comprise any of the CAR polynucleotides described herein. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides can be synthesized in situ in the cell after introduction of polynucleotides encoding the CAR polypeptides into the cell. Alternatively, CAR polypeptides can be produced outside of cells, and then introduced into cells. Methods for introducing a polynucleotide construct into cells are known in the art. In some embodiments, stable transformation methods (e.g., using a lentiviral vector) can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments, isolated nucleic acids are provided comprising a promoter operably linked to a first polynucleotide encoding an antigen binding domain, at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

In some embodiments, the isolated nucleic construct is contained within a viral vector and is introduced into the genome of an engineered immune cell by random integration, e.g., lentiviral- or retroviral-mediated random integration. In some embodiments, the isolated nucleic acid construct is contained in a viral vector or a non-viral vector and is introduced into the genome of an engineered immune cell by site-specific integration, e.g., adenovirus-mediated site-specific integration.

### 3. Manufacture of Engineered Immune Cells (Including CAR T Cells)

Provided herein are methods of analyzing or determining various attributes of donor cells from a donor cell population and/or engineered immune cells from a population of immune cells (including engineered immune cells such as CAR expressing or CAR+ cells). As described herein, engineered immune cells, such as CAR T cells, can be modified to reduce or eliminate expression or activity of endogenous TCR, and the remaining TCR+ engineered immune cells can be depleted according to the methods described herein, at the end of production. The instant disclosure provides methods of characterizing or analyzing a population of engineered immune cells to characterize the drug product or as part of the manufacturing process. The instant disclosure also provides methods of analyzing or determining other attributes, such as the potency or polyfunctionality of the engineered immune cells to characterize the drug product or as part of the manufacturing process. In some embodiments, the engineered immune cells, such as CAR T cells, are manufactured according to Good Manufacturing Practice (GMP).

A variety of known techniques can be utilized in making the polynucleotides, polypeptides, vectors, antigen binding domains, immune cells, compositions, and the like according to the disclosure.

Prior to the in vitro manipulation or genetic modification of the immune cells described herein, the cells can be obtained from a subject. Cells expressing a CAR can be derived from an allogeneic or autologous source and can be depleted of endogenous TCR as described herein.

a. Source Material

In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In certain some embodiments, the cells collected by apheresis can be washed to remove the plasma fraction, and then placed in an appropriate buffer or media for subsequent processing.

In certain some embodiments, T cells are isolated from PBMCs by lysing the red blood cells and depleting the monocytes, for example, using centrifugation through a PERCOLL™ gradient. A specific subpopulation of T cells, (e.g., CD28+, CD4+, CD45RA−, and CD45RO+T cells or CD28+, CD4+, CDS+, CD45RA−, CD45RO+, and CD62L+ T cells) can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion. In some embodiments, CD8+ cells are further sorted into naive, stem cell memory, central memory, and effector cells by identifying cell surface antigens that are associated with each of these types of CD8+ cells. In some embodiments, the expression of phenotypic markers of central memory T cells include CD27, CD45RA, CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin. In certain some embodiments, CD4+ T cells are further sorted into subpopulations. For example, CD4+ T helper cells can be sorted into naive, central memory, and effector cells by identifying cell populations that have cell surface antigens.

b. Stem Cell Derived Immune Cells

In some embodiments, the immune cells can be derived from embryonic stem (ES) or induced pluripotent stem (iPS) cells. Suitable HSCs, mesenchymal, iPS cells and other types of stem cells can be cultivated immortal cell lines or isolated directly from a patient. Various methods for isolating, developing, and/or cultivating stem cells are known in the art and can be used to practice the present disclosure.

In some embodiments, the immune cell is an induced pluripotent stem cell (iPSC) derived from a reprogrammed T-cell. In some embodiments, the source material can be an induced pluripotent stem cell (iPSC) derived from a T cell or a non-T cell. The source material can be an embryonic stem cell. The source material can be a B cell, or any other cell from peripheral blood mononuclear cell isolates, hematopoietic progenitor, hematopoietic stem cell, mesenchymal stem cell, adipose stem cell, or any other somatic cell type.

c. Genetic Modification of Isolated Cells

The donor immune cells, e.g., T cells, of a donor immune cell population can be genetically modified following isolation using known methods, or the donor immune cells can be activated and expanded (or differentiated in the case of progenitors) in vitro prior to being genetically modified. In some embodiments, the isolated donor immune cells are genetically modified to reduce or eliminate expression or activity of endogenous TCRα and/or CD52. In some embodiments, the cells are genetically modified using gene editing technology (e.g., CRISPR/Cas9, CRISPR/Cas12a, a zinc finger nuclease (ZFN), a TALEN, a MegaTAL, a meganuclease) to reduce or eliminate expression or activity of endogenous proteins (e.g., TCRα and/or CD52). In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro.

Following genetic modification to reduce or eliminate expression or activity of an endogenous gene or genes, e.g., endogenous TCRα and/or CD52, to provide engineered immune cells derived from the donor cells, the engineered immune cells may be depleted (according to the methods described herein) of engineered immune cells that expression one or more unwanted biomarkers, e.g., one or more of HLA-DR, TIGIT, CD16, CD56, and any combination thereof. FIG. 5 depicts a workflow for the manufacture of engineered immune cells including a depletion step following the reduction/elimination of expression/activity step for an endogenous gene (Post-Gene Knock Out Depletion based on Biomarker Profiling). A depletion step based on biomarker profiling may be performed at additional time points, such as prior to activation or after activation

Certain methods for making the constructs and engineered immune cells of the disclosure are described in PCT application PCT/US15/14520, the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In certain some embodiments, the present disclosure provides isolated host cells containing the vector provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells, particularly human cells.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs or TCRs. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

d. Allogeneic CAR T Cells

The process for manufacturing allogeneic CAR T therapy involves harvesting healthy, selected, screened and tested donor immune cells, including T cells, from healthy donors. Next, the T cells of the donor immune cells are engineered to express CARs, which recognize certain cell surface proteins that are expressed in hematologic or solid tumors. Allogeneic T cells are gene editing to reduce the risk of graft versus host disease (GvHD) and to prevent allogeneic rejection. A T cell receptor gene (e.g., TCRα, TCRβ) is knocked out to avoid GvHD. The CD52 gene can be knocked out to render the CAR T product resistant to anti-CD52 antibody treatment. Anti-CD52 antibody treatment can therefore be used to suppress the host immune system and allow the CAR T to stay engrafted to achieve full therapeutic impact. The engineered T cells then undergo further processing, which may optionally include a depletion step to remove unwanted T cells that express a biomarker profile described herein (e.g., unwanted T cells expressing one or more of HLA-DR, TIGIT, CD16, CD56, and any combination thereof), as well as a purification step and are ultimately cryopreserved in vials for delivery to patients.

e. Autologous CAR T Cells

Autologous chimeric antigen receptor (CAR) T cell therapy, involves collecting a patient's own cells (e.g., white blood cells, including T cells) and genetically engineering the T cells to express CARs that recognize target expressed on the cell surface of one or more specific cancer cells and kill cancer cells. The engineered T cells may optionally be subjected to a depletion step to remove unwanted T cells that express a biomarker profile described herein (e.g., unwanted T cells expressing one or more of HLA-DR, TIGIT, CD16, CD56, and any combination thereof). The engineered cells are then cryopreserved and subsequently administered to the patient.

### 4. Methods of In Vitro Sorting

In some embodiments, provided are methods for in vitro sorting of a population of immune cells, wherein a subset of the population of immune cells comprises engineered immune cells expressing an antigen-specific CARs comprising epitopes specific for monoclonal antibodies (e.g., exemplary mimotope sequences). The method comprises contacting the population of immune cells with a monoclonal antibody specific for the epitopes and selecting the immune cells that bind to the monoclonal antibody to obtain a population of cells enriched in engineered immune cells expressing an antigen-specific CAR.

In some embodiments, said monoclonal antibody specific for said epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10. In other embodiments the mAb binds to TCRα or TCRβ.

In some embodiments, the population CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30%, 40% or 50%. In some embodiments, the immune cells are T-cells.

In some embodiments, the mAbs are previously bound onto a support or surface. Non-limiting examples of solid support can include a bead, agarose bead, a magnetic bead, a plastic welled plate, a glass welled plate, a ceramic welled plate, a column, or a cell culture bag.

The CAR-expressing immune cells to be administered to the recipient can be enriched in vitro from the source population. Methods of expanding source populations can include selecting cells that express an antigen such as CD34 antigen, using combinations of density centrifugation, immuno-magnetic bead purification, affinity chromatography, and fluorescent activated cell sorting.

Flow cytometry can be used to quantify specific cell types within a population of cells. In general, flow cytometry is a method for quantifying components or structural features of cells primarily by optical means. Since different cell types can be distinguished by quantifying structural features, flow cytometry and cell sorting can be used to count and sort cells of different phenotypes in a mixture.

In some embodiments, the method used for sorting T cells expressing CAR is the Magnetic-Activated Cell Sorting (MACS). Magnetic-activated cell sorting (MACS) is a method for separation of various cell populations depending on their surface antigens (e.g., CD molecules) by using superparamagnetic nanoparticles and columns. MACS can be used to obtain a pure cell population. Cells in a single-cell suspension can be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

Detailed protocol for the purification of specific cell population such as T-cell can be found in Basu S et al. (2010). (Basu S, Campbell H M, Dittel B N, Ray A. Purification of specific cell population by fluorescence activated cell sorting (FACS). J Vis Exp. (41): 1546).

### 5. Pharmaceutical Compositions and Therapy

In some embodiments, the engineered immune cells described herein are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

In embodiments, desired treatment amounts of cells in the composition are generally at least 2 cells (for example, at least 1 CD8+ central or stem cell memory T cell and at least 1 CD4+ helper T cell subset; or two or more CD8+ central or stem cell memory T cell; or two or more CD4+ helper T cell subset) or is more typically greater than 10² cells, and up to and including 10⁶, up to and including 10⁷, 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of about 10⁵/kilogram or about 10⁶/kilogram (10⁶-10¹¹ per patient) can be administered. CAR treatments can be administered multiple times at dosages within these ranges. The cells can be autologous, allogeneic, or heterologous to the patient undergoing therapy.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

### 6. Methods of Treatment

The disclosure comprises methods for treating or preventing a disease (e.g., cancer) in a patient, comprising administering to a patient in need thereof an effective amount of engineered immune cells (e.g., CAR T cells, or engineered immune cells comprising a CAR disclosed herein) that have been derived from donor cells having a biomarker profile as described herein. In some embodiments, the effective amount of CAR T cells or engineered immune cells have been analyzed for various attributes according to the methods described in the instant disclosure. In some embodiments, the CAR T cell drug product for therapeutic use has been analyzed for various attributes, such as a certain biomarker profile, potency or polyfunctionality according to the methods described in the instant disclosure. In some embodiments, the CAR T cells are TCR− CAR T cells, and the CAR T drug product for therapeutic use has been analyzed for various attributes, such as the amount or percentage of remaining TCR+ CAR T cells and/or potency or polyfunctionality according to the methods described in the instant disclosure.

Methods are provided for treating diseases or disorders, including cancer. In some embodiments, the disclosure relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR). In some embodiments, the target cell is a tumor cell. In some aspects, the disclosure comprises a method for treating or preventing a malignancy, said method comprising administering to a subject in need thereof an effective amount of at least one isolated antigen binding domain described herein. In some aspects, the disclosure comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding domain as described herein. The CAR containing immune cells of the disclosure can be used to treat malignancies involving aberrant expression of biomarkers. In some embodiments, CAR containing immune cells of the disclosure can be used to treat small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features. In exemplary embodiments, the CAR containing immune cells, e.g., CAR-T cells of the disclosure are used to treat small cell lung cancer.

Also provided are methods for reducing the size of a tumor in a subject, comprising administering to the subject an engineered cell of the present disclosure to the subject, wherein the cell comprises a chimeric antigen receptor comprising an antigen binding domain and binds to an antigen on the tumor.

In some embodiments, the subject has a solid tumor, or a blood malignancy such as lymphoma or leukemia. In some embodiments, the engineered cell is delivered to a tumor bed. In some embodiments, the cancer is present in the bone marrow of the subject. In some embodiments, the engineered cells are autologous immune cells, e.g., autologous T cells. In some embodiments, the engineered cells are allogeneic immune cells, e.g., allogeneic T cells. In some embodiments, the engineered cells are heterologous immune cells, e.g., heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo.

A “therapeutically effective amount,” “effective dose,” “effective amount,” or “therapeutically effective dosage” of a therapeutic agent, e.g., engineered CART cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Desired treatment amounts of cells in the composition is generally at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of 10⁶/kilogram (10⁶-10¹¹ per patient) can be administered. CAR treatments can be administered multiple times at dosages within these ranges. The cells can be autologous, allogeneic, or heterologous to the patient undergoing therapy.

In some embodiments, the therapeutically effective amount of the CAR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, target doses for CAR+/CAR-T+/TCR+ cells range from 1×10⁶-2×10⁸ cells/kg, for example 2×10⁶ cells/kg. It will be appreciated that doses above and below this range can be appropriate for certain subjects, and appropriate dose levels can be determined by the healthcare provider as needed. Additionally, multiple doses of cells can be provided in accordance with the disclosure.

In some aspect, the disclosure comprises a pharmaceutical composition comprising at least one antigen binding domain as described herein and a pharmaceutically acceptable excipient. In some embodiments, the pharmaceutical composition further comprises an additional active agent.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

In some embodiments, upon administration to a patient, engineered immune cells expressing at their cell surface any one of the antigen-specific CARs described herein can reduce, kill or lyse endogenous antigen-expressing cells of the patient. In one embodiment, a percentage reduction or lysis of antigen-expressing endogenous cells or cells of a cell line expressing an antigen by engineered immune cells expressing any one of an antigen-specific CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of antigen-expressing endogenous cells or cells of a cell line expressing an antigen by engineered immune cells expressing antigen-specific CARs is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%. In one embodiment, the endogenous antigen-expressing cells are endogenous antigen-expressing bone marrow cells.

In one embodiment, the percent reduction or lysis of target cells, e.g., a cell line expressing an antigen, by engineered immune cells expressing at their cell surface membrane an antigen-specific CAR of the disclosure can be measured using the assay disclosed herein.

The methods can further comprise administering one or more chemotherapeutic agent. In certain some embodiments, the chemotherapeutic agent is a lymphodepleting (preconditioning) chemotherapeutic. For example, methods of conditioning a patient in need of a T cell therapy comprising administering to the patient specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). A preferred dose regimen involves treating a patient comprising administering daily to the patient about 300 mg/m²/day of cyclophosphamide and about 30 mg/m²/day of fludarabine for three days prior to administration of a therapeutically effective amount of engineered T cells to the patient.

In some embodiments, lymphodepletion further comprises administration of a CD52 antibody. In some embodiments, the CD52 antibody is alemtuzumab. In some embodiments, the CD52 antibody is administered at a dose of about 13 mg/day IV.

In other embodiments, the antigen binding domain, transduced (or otherwise engineered) cells and the chemotherapeutic agent are administered each in an amount effective to treat the disease or condition in the subject.

In certain some embodiments, compositions comprising CAR-expressing immune effector cells disclosed herein can be administered in conjunction with any number of chemotherapeutic agents, which can be administered in any order. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RF S2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further comprise administering two or more chemotherapeutic agents.

A variety of additional therapeutic agents can be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab (Tcentriq®).

Additional therapeutic agents suitable for use in combination with the disclosure include, but are not limited to, ibrutinib (Imbruvica®), ofatumumab (Arzerra®, rituximab (Rituxan®), bevacizumab (Avastin®), trastuzumab (Herceptin®), trastuzumab emtansine (KADCYLA®, imatinib (Gleevec®), cetuximab (Erbitux®, panitumumab) (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR-containing immune cells can be administered with a therapeutic regimen to prevent cytokine release syndrome (CRS) or neurotoxicity. The therapeutic regimen to prevent cytokine release syndrome (CRS) or neurotoxicity can include lenzilumab, tocilizumab, atrial natriuretic peptide (ANP), anakinra, iNOS inhibitors (e.g., L-NIL or 1400W). In additional embodiments, the composition comprising CAR-containing immune cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (Ils) such as IL-1, IL-1alpha, IL-2, IL-3, IL-4, IL-5, IL-6, IL-7, IL-8, IL-9, IL-10, IL-11, IL-12; IL-15, IL-21 a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### 7. Kits and Articles of Manufacture

The present disclosure provides kits comprising reagents for analyzing cell populations described herein, including donor cell populations and engineered cell populations, including CAR T drug products. In some embodiments, the kit comprises one or more reagents for the detection of one or more biomarkers including HLA-DR, TIGIT, CD16, CD56, CD27, CCR7, and CD45RA. The reagents may be antigen binding molecules having specificity, such as an antibody as described herein, to the one or more biomarkers. In some embodiments, the kit further comprises one or more reagents for the detection of the CAR. In some embodiments, the kit further comprises one or more reagents for the detection of TCRαβ. In some embodiments, the kit comprises one or more reagent for analyzing the cell populations described herein according to the methods described herein, wherein the one or more reagent is conjugated with a detection label. The kit may also comprise instructions on the use of the reagents to detect levels of the one or more biomarkers, wherein the detected level indicates a percentage of expression for one or more biomarkers described herein. In another embodiment, the kit may further comprise reagents for measuring the in vitro functionality of an engineered immune cell, e.g., a CAR T cell. The measured in vitro functionality may comprise in vitro cytotoxicity, mitochondrial fitness, and/or cytokine secretion profiling.

The present disclosure also provides kits comprising any of the cultured immune cells or engineered immune cells described herein, and pharmaceutical compositions of the same. In some exemplary embodiments, a kit of the disclosure comprises allogeneic CAR T cells for administering to a subject.

The present application further provides articles of manufacture comprising any one of the therapeutic compositions or kits described herein. Examples of an article of manufacture include vials (e.g. sealed vials).

The following examples are offered for illustrative purposes only. Indeed, various modifications of the invention in addition to those shown and described herein will become apparent to those skilled in the art from the foregoing description.

### EXAMPLES

### Example 1—Biomarker Characterization of Donor Cell Populations

Peripheral blood mononuclear cells (PBMC) and CD4+ CD8+ cells enriched from PBMC were surface-stained with T cell phenotypic, exhaustion, activation markers and markers to differentiate different cell types. Data from stained cells were acquired using BD LSR Fortessa X-20. Difference between donors can be characterized with flow cytometry and younger donors tend to have higher expression of young-T cell phenotypic markers (e.g., C—C Chemokine Receptor 7 (CCR7), CD45RA etc.) and lower expression of exhaustion markers (e.g., TIGIT). Other expression marker levels that were observed include human leukocyte antigen-DR isotype (HLA-DR), CD27, CD16, and CD56.

In addition, CAR-T cells derived from donor cells as per the process described in Example 2 were similarly stained to detect different markers at the end of the CAR-T manufacturing process. Table 1A provides the percentage range of cells found to express each biomarker in the CAR-T cell populations that were tested. The CAR-T cell populations were tested for in vitro toxicity using the long-term killing assay (LTKA) as described in Example 2. Table 1A provides an exemplary percentage of biomarker expression in the CAR-T cells which correlates to improved in vitro cytotoxicity. For example, a CAR-T cell population having less than 65% cells expressing HLA-DR exhibited stronger in vitro cytotoxicity than a CAR-T cell population having more than 65% cells expressing HLA-DR.

TABLE 1A

| Biomarker | Range detected | Exemplary percentage |
|---|---|---|
| HLA-DR | 38.1 -98.1% | <65 |
| TIGIT | 3.41 - 70.7% | <30 |
| CD16 | 02. - 27.3% | <4 |
| CD56 | 4.43 - 26% | <15 |
| CD27 | 17.2 -91.8% | >55 |
| CCR7 | 2.73 - 63.5% | >30 |
| CD45RA | 30.9 - 92.5% | >70 |

### Example 2—In Vitro Functional Analysis of CAR T Cells Derived from Donor Cell Populations

The donor cell populations described in Example 1 were used to engineer CAR T cells targeting B-cell maturation antigen (BCMA) (e.g., as described in Kuo et al. U.S. Pat. No. 10,294,304, such as Example 2, which is incorporated herein by reference in its entirety). Briefly, CAR T cells were manufactured from a healthy donor PBMCs in a process involving the transduction of lentivirus harboring the CAR scFv transgene recognizing BCMA. Cells were then seeded and activated with TransAct™ activation agent (MACS GMP T cell TransAct™) to induce activation and proliferation of the T cells. Cells were then plated in six-well plates with the LVV containing the construct that expresses the BCMA scFv/4-1BB/CD3t CAR. Then, TALEN® mRNAs were transfected into T cells by electroporation (EP) to genetically disrupt TRAC and CD52 genes and the TCRαβ and CD52 protein expression using the Lonza™ 4D-Nucleofector system. At the end of manufacturing, CAR T cells were frozen in CryoStor® CS5 solution and stored in liquid nitrogen freezer for further functional characterization.

The CAR T cells were then analyzed using various in vitro functional assays including cytotoxicity, mitochondrial fitness, and cytokine secretion profiling.

Cytotoxicity. A short-term killing assay (STKA) and a long-term killing assay (LTKA) were used to determine CAR T cell cytotoxicity.

STKA. Frozen CAR T cells and non-transduced cells were thawed, and viability and cell count were checked with Vi-Cell XR analyzer from Beckman Coulter. The % CAR+ of different donors were normalized with non-transduced cells so that all donors have equal % CAR+ and total number of T cells. Assay was set up in 96-well flat-bottom plate with the same number of MM1s-GFP-Luc cells as target cells and alter the T cell number to achieve effector (CAR T cells) to target cell ratio as 10:1, 5:1, 2.5:1, 1.25:1, 0.625:1, 0.3:1 and 0.15:1. Triplicate wells were prepared for each condition and plate were kept in 37° C., CO2 supplement incubator after setup. After overnight incubation (about 24 hours), cytotoxicity was measured by measuring luciferase signal using Promega Bright-Glo Luciferase Assay System on Promega GloMax instrument.

LTKA. Frozen CAR T cells and non-transduced cells were thawed, and viability and cell count were checked with Vi-Cell XR analyzer from Beckman Coulter. The % CAR+ of different donors were normalized with non-transduced cells so that all donors have equal % CAR+ and total number of T cells. Assay was set up in 96-well flat-bottom plate with the same number of MM1s-GFP-Luc cells as target cells and alter the T cell number to achieve effector (CAR T cells) to target cell ratio as 2:1, 1:1 or 1:2. Quadruplicate wells were prepared for each condition and plate were kept in 37° C., CO2 supplement incubator after setup. Every 2-3 days, half of the cells were transferred to a new 96-well flat-bottom plate and re-challenged with the same amount of target cells as setup to evaluate long-term cytotoxicity and persistency whereas the other half of the cells were used to measure cytotoxicity but measuring luciferase signal using Promega Bright-Glo Luciferase Assay System on Promega GloMax instrument. Assay were continued until no cytotoxicity were observed.

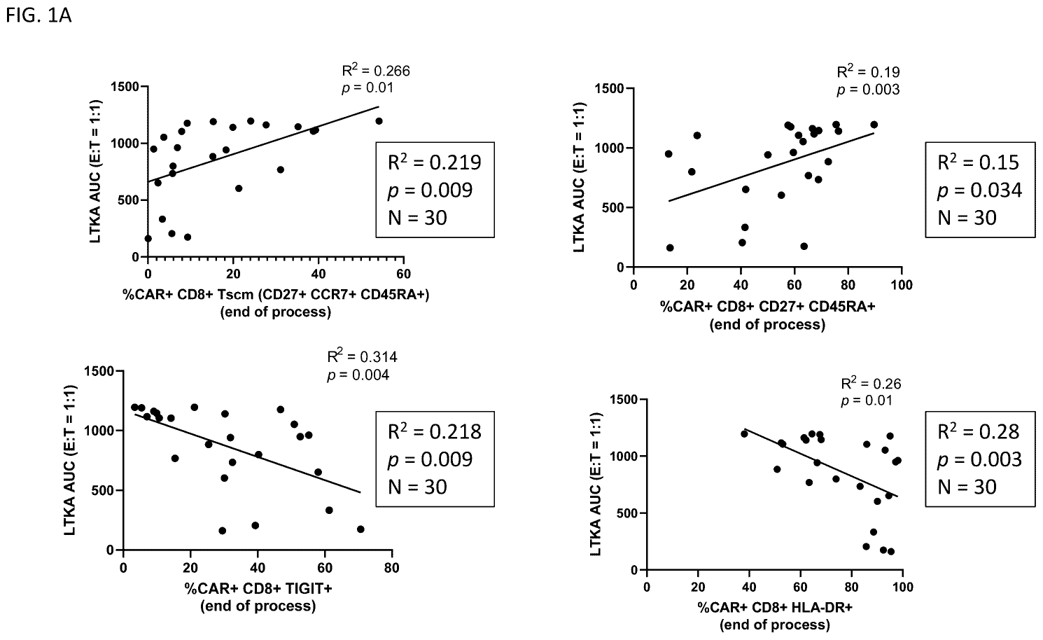

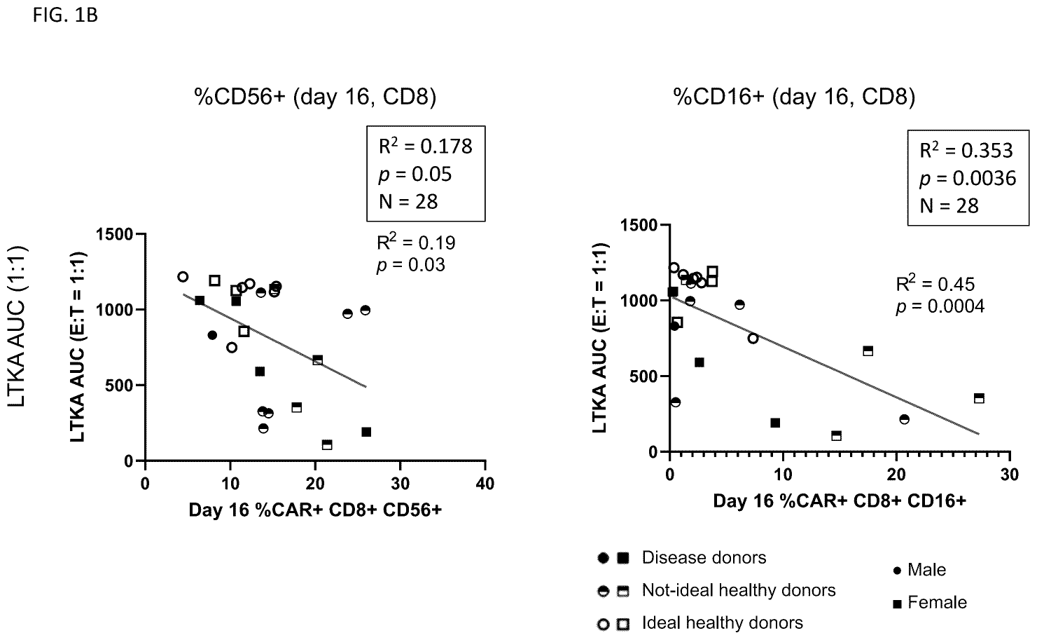

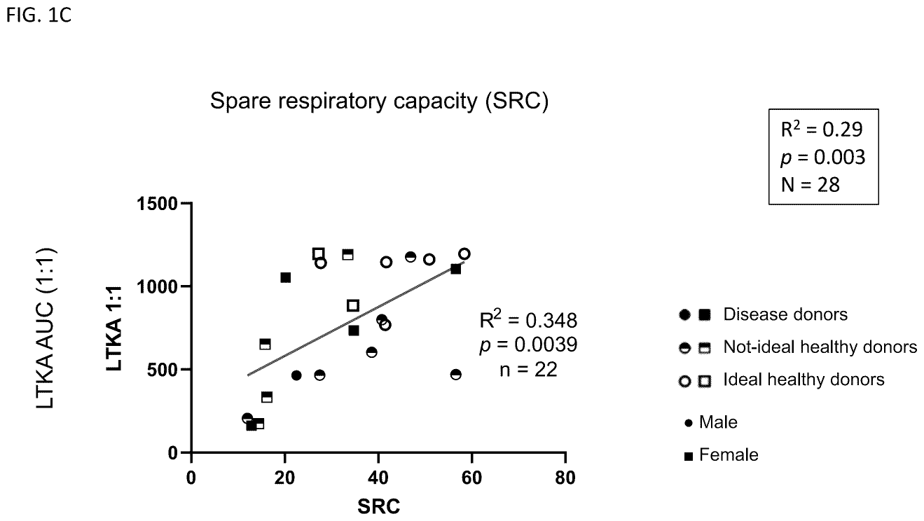

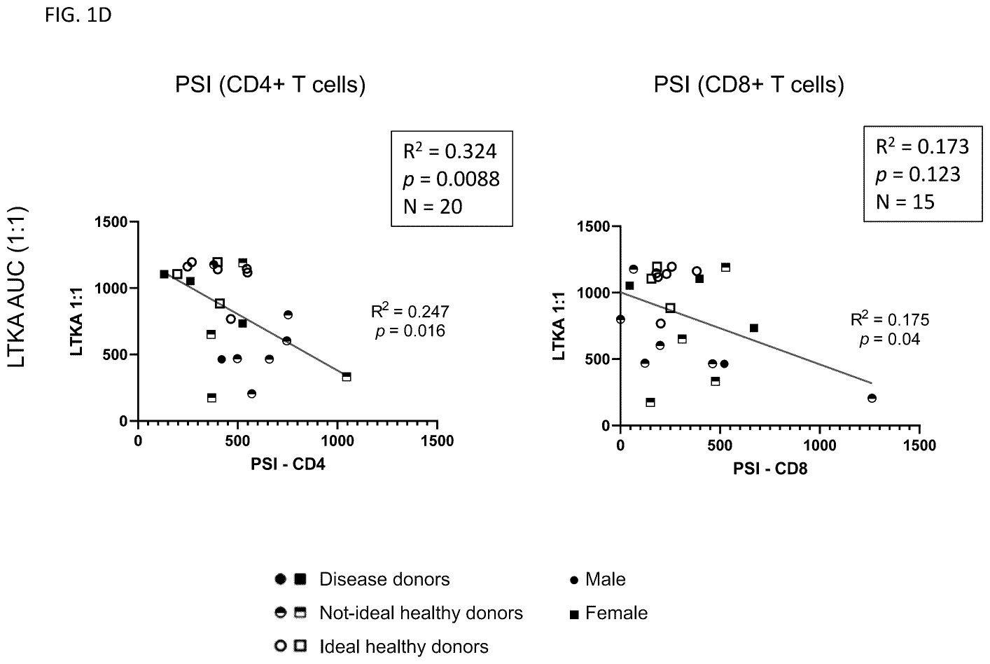

FIG. 1A-1B depicts associations between in vitro CAR-T functionality (long-term killing assay or LTKA) and certain biomarkers on CD8+ CAR-T cells at the end of CAR-T cell manufacturing. FIG. 1A depicts an observed positive association between in vitro CAR-T functionality and expression of (i) CD27, CCR7, and CD45RA (top left panel) or (ii) CD27 and CD45RA (top right panel). FIG. 1A also depicts (i) an observed negative association between in vitro CAR-T functionality and the expression of (i) T cell immunoreceptor with Ig and immunoreceptor tyrosine-based inhibitory motif (ITIM) domains (TIGIT) (lower left panel) or and (ii) human leukocyte antigen-DR isotype (HLA-DR) (lower right panel) on T cells at the end of CAR T cell manufacturing. FIG. 1B depicts an observed negative association between in vitro CAR T functionality and the expression of CD56 or CD16 on CD8+ CAR-T cells at the end of CAR T cell manufacturing. Expression of early T cell phenotypic markers and exhaustion markers were found to trend with in vitro anti-tumor activity. Additional donors were evaluated for LTKA associations and the following observations were made: i) a positive association between LTKA and percentage of CD27, CCR7, and CD45RA triple positive population—R²=0.219, p=0.009, and N=30; ii) a positive association between LTKA and percentage of CD27 and CD45RA double positive population—R²=0.15, p=0.034, and N=30; iii) a negative association between LTKA and expression of TIGIT—R²=0.218, p=0.009, and N=30; iv) a negative association between LTKA and expression of HLA-DR—R²=0.28, p=0.003, and N=30; v) a negative association between LTKA and expression of CD56—R²=0.178, p=0.05, and N=28; and vi) a negative association between LTKA and expression of CD16—R²=0.353, p=0.0036, and N=28.

Mitochondrial fitness. The mitochondrial fitness of CAR T cells was characterized with Agilent Seahorse XF Cell Mito Stress Test Kit. Frozen CAR T cells were thawed, and viability and cell count were checked with Vi-Cell XR analyzer from Beckman Coulter. Cell concentrations were adjusted to 1×10⁶ cells/ml in R10 media (RPMI+10% HI-FBS+1× non-essential amino acid+1× sodium pyruvate) and rest in 37° C., CO₂ supplement incubator for at least 1 hour. After incubation, assay was conducted by following the manufacturer's protocol and data was collected using Seahorse XFe96 Analyzer. Spare respiratory capacity (SRC) was measured with the instrument indicating how much extra energy cells can provide upon emergency and higher SRCs being indicative of cells with fitter mitochondrial function. It was observed that the SRC value positively trends with cytotoxicity results from LTKA (FIG. 1C) and younger healthy donors also have slightly higher SRC compared to elder donors, indicating potential relationship between mitochondrial fitness with donor characteristics.

Cytokine secretion profiling. Cytokine secretion at the single-cell level was measured to generate a polyfunctional strength index (PSI) (Isoplexis Isocode technology) from donor cell starting material or thawed CAR T cells. Briefly, CD4/CD8-enriched cells from PBMCs (CD3+) were stimulated with SEB or TransAct. On day zero, the CD4/CD8-enriched cells were isolated from PBMCs, stimulant was added and incubated overnight. On day 1, the cells were then stained with Stain A and loaded onto chips. Data was collected on IsoLight. Using the formula below, PSI measured the cytokine secretion capacity at single cell level and the intrinsic differences between donors in cytokine secretion were observed with this assay.

$$PSI = ( \% polyfunctionality ) {\sum }_{i = 1}^{32} {MFI}_{i}$$

In another experiment, thawed CAR T cells were separated by CD4/8 expression (CD4+ or CD8+) and stimulated with target cells (MM1s). On day zero, CAR T cells were thawed, recovered in 1 M/ml concentration with IL-2 overnight. On day 1, CD4+ and CD8+ cells were separated, co-incubated with target cells at E:T ratio of 1:2 overnight. On day 2, T cells were separated from target cells, stained with CD4− or CD8− AF647 and loaded on chips. Data was collected on IsoLight. Similar to the results above, different donors have a range of cytokine secretion capacity. However, single-cell cytokine secretion potency negatively trends with T cell cytotoxicity. (FIG. 1D). Without being bound by theory, one explanation for these results is that cells may have limited energy under stressed conditions and there is competition between cytokine production and cytotoxicity, thus, leading to the negative trends between the two assays. One interesting observation is that younger healthy donors tend to have lower PSI compared to healthy donors with age >30 in both CAR T cells and starting material. Although the difference is not significant, this may be one attribute for further analysis to help advance an understanding of donor differences. In summary, different donor cell populations were used to generate CAR T cell populations, which were then analyzed for certain biomarkers and functional attributes. Table 1B summarizes the results using cells from different types of donor populations: i) healthy, ideal and ii) cancer patients.

In addition to the median (50%) value, the Q1 (25%) and Q3 (75%) values are provided for the data ranges between the minimum and maximum values. These Q1 and Q3 values provide additional insight when comparing healthy ideal donors to cancer patients. For example, the median and range (maximum minus minimum value) differences in STKA AUC are not as apparent between healthy ideal donors and cancer patients; but Q1 and Q3 values of healthy ideal donors are consistently higher than cancer patients. The additional Q1 and Q3 values provide information on the distribution of data and showed the overall superior cytotoxicity of healthy ideal donors. In the case of PSI of CD8+ CAR T cells, which showed negative correlation with LTKA efficacy, despite the lower minimum value in cancer patients, the addition of Q1 information showed the higher trend of PSI value in cancer patients and provided more insight into the data distribution.

TABLE 1B

| | | | | | | | | PSI | PSI |
|---|---|---|---|---|---|---|---|---|---|
| | | | | | | | | (SEB, | (TransAct, |
| | | | LTKA | | | PSI | PSI | day 0 | day 0 |
| Donor | | | AUC | STKA | | (CD4 | (CD8 | starting | starting |
| Type | Statistics | Age | (E:T = 1:1) | AUC | SRC | CAR T) | CAR T) | material) | material) |
| Healthy | Median | 22 | 1146 | 444.9 | 41.5142 | 399.77 | 194.975 | 43.82 | 29.38 |
| ideal | Minimum | 19 | 749 | 209.4 | 27.1517 | 197.38 | 155.43 | 0 | 0 |
| | Maximum | 29 | 1217 | 518.1 | 58.4044 | 548.4 | 382.88 | 258.38 | 195.61 |
| | Q1 | 20.5 | 1117 | 409 | 31.1246 | 269.95 | 182.54 | 9.43 | 5.56 |
| | Q3 | 27 | 1170 | 470.3 | 46.3018 | 465.57 | 237.75 | 49.51 | 97.09 |
| Cancer | Median | 56 | 830.7 | 326.1 | 22.4837 | 340.775 | 459.425 | 66.38 | 122.21 |
| patients | Minimum | 28 | 190 | 200.8 | 12.8612 | 131.03 | 46.58 | 29.11 | 62.21 |
| | Maximum | 72 | 1060 | 534.7 | 56.636 | 524.55 | 669.23 | 115.17 | 215 |
| | Q1 | 38.25 | 640.278 | 306.1 | 19.1776 | 229.34 | 309.763 | 47.745 | 92.21 |
| | Q3 | 65.25 | 1010.5 | 365.2 | 37.7501 | 445.718 | 558.328 | 90.775 | 168.605 |

### Example 3—Allogeneic CAR T Cells Derived from Younger Donor T Cells have More Desirable T Cell Phenotype and Better In Vitro Functionality

Autologous chimeric antigen receptor (CAR) T cell therapy has shown promising efficacy in treating relapsed/refractory B cell malignancies. Despite clinical success, autologous CAR T cell therapy has disadvantages including delays in treating patients and the inability to treat all patients due to manufacturing failures stemming from dysfunctional T cells present in this patient population. In contrast, investigational allogeneic CAR T cell therapy uses T cells from healthy individuals as starting material, simplifying supply, and providing off-the-shelf product convenience. Using healthy donors T cells also opens the possibility to optimize therapeutic efficacy by using donor T cells that are immunologically fit and provide a more homogeneous product. Given that most CAR T cell performance assessments have been conducted with patient-derived CAR T cells, what constitutes an unfit donor for allogeneic T cell therapy is still unclear.

To understand factors influencing donor suitability, this study evaluated T cells from 19 healthy donors. The healthy donors chosen were diverse in age (range: 18-62) and body mass index (BMI) (range: 19-52) to capture a broad spectrum of physical fitness and evaluate the impact of age and BMI on CAR T cell phenotype and function. In addition, T cells from 11 donors with relapsed/refractory heme malignancies, referred to here as patient-derived T cells, were also included in the donor pool as a control for dysfunctional T cells, emulating autologous CAR T cell therapies.

T cells were isolated from the peripheral blood mononuclear cells (PBMC) of these 30 donors and used as starting material to generate CAR T cells. During the CAR T cell production in the lab, 6 out of 11 patient-derived T cell preparations failed to expand due to the limited number of viable and fit T cells in the staring material. CAR T cells were successfully generated, however, from the remaining 24 donors (19 healthy-donor and 5 patient-derived T cell preparations).

These CAR T cell batches were characterized further with an array of in vitro assays, including deep immunophenotyping by flow cytometry and cytotoxicity assays. Correlational analyses revealed a negative correlation between in vitro anti-tumor activity and increased age of the donor.

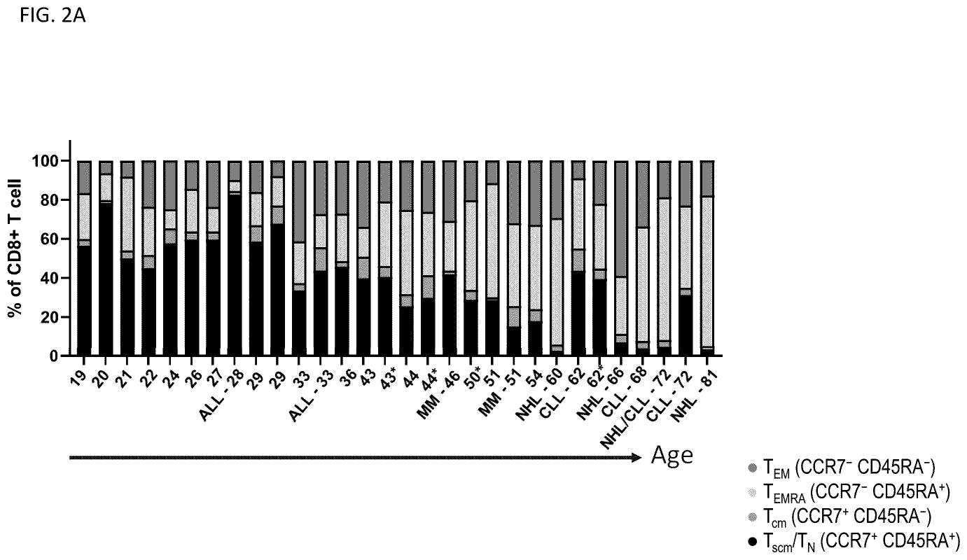

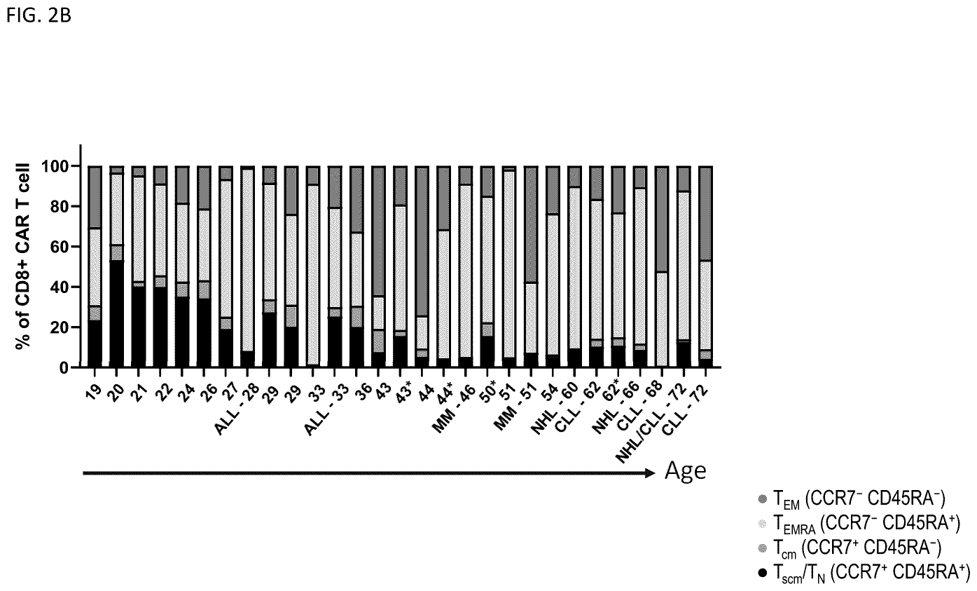

Furthermore, there was a negative correlation between the percentage of less differentiated T cells in both the starting material and the CAR T cell product and age, with older donors having less stem/central memory T cells than younger donors as shown in FIG. 2A (CD8 T cell phenotype—starting material) and FIG. 2B (CD8 T cell phenotype after CAR T manufacturing). An asterisk (*) indicates a non-ideal donors having a BMI of >30. Diseased donors (providing patient-derived T cells) are labeled with the disease (ALL: acute lymphoblastic leukemia; MM: multiple myeloma; CLL: chronic lymphocytic leukemia and NHL: Non-Hodgkin lymphoma).

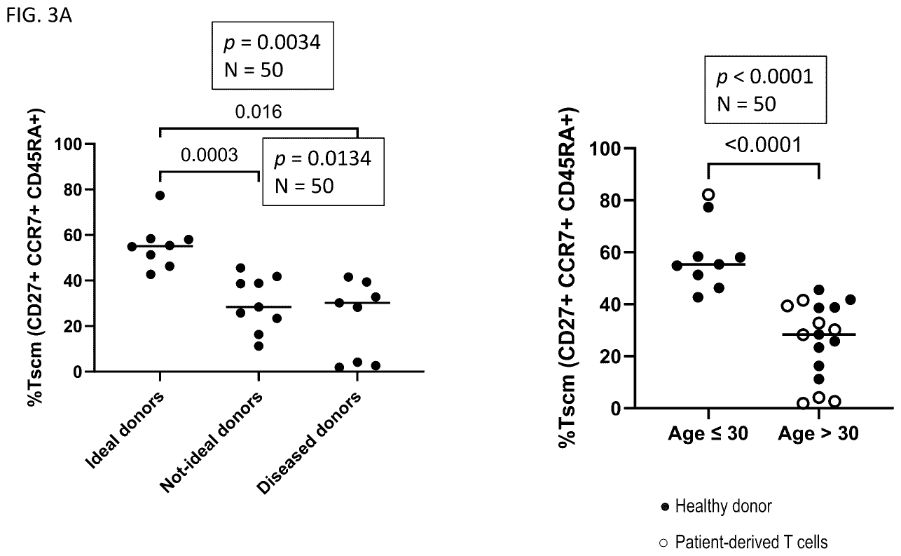

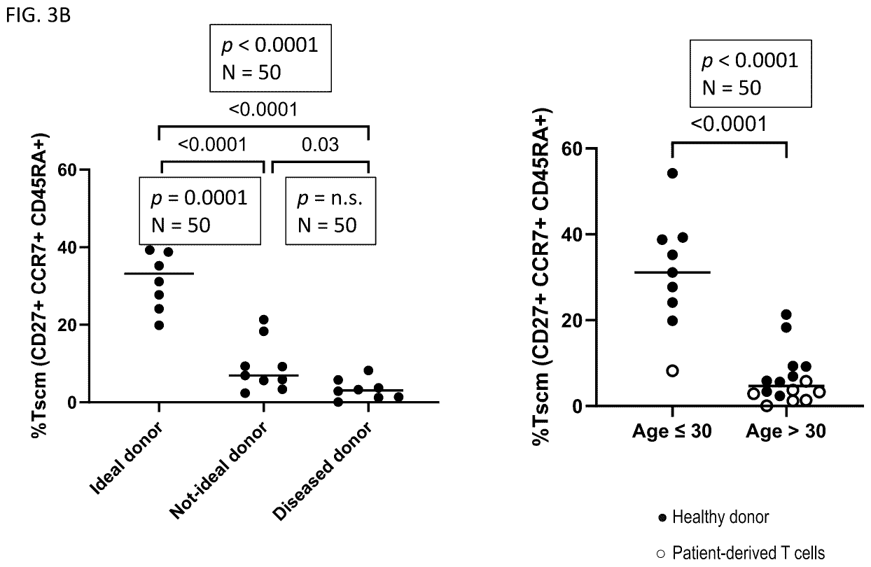

Despite having a similar percentage of stem cell memory T cells (TSCM) in the starting material as age-matched healthy donors, patient-derived CAR T cells also tended to have a lower percentage of TSCM at the end of culture compared to CAR T cells generated from healthy donor material, highlighting the limited fitness of disease donor T cells (FIG. 3A—starting material and FIG. 3B—after CAR T manufacturing). Both donor age and disease state were found to be associated with lower % Tscm and this difference increases during CAR T manufacturing. Donor age and disease state both contribute to the lower % Tscm in the starting material. In addition, disease state is also associated with lower % Tscm at the end of CAR T manufacturing.

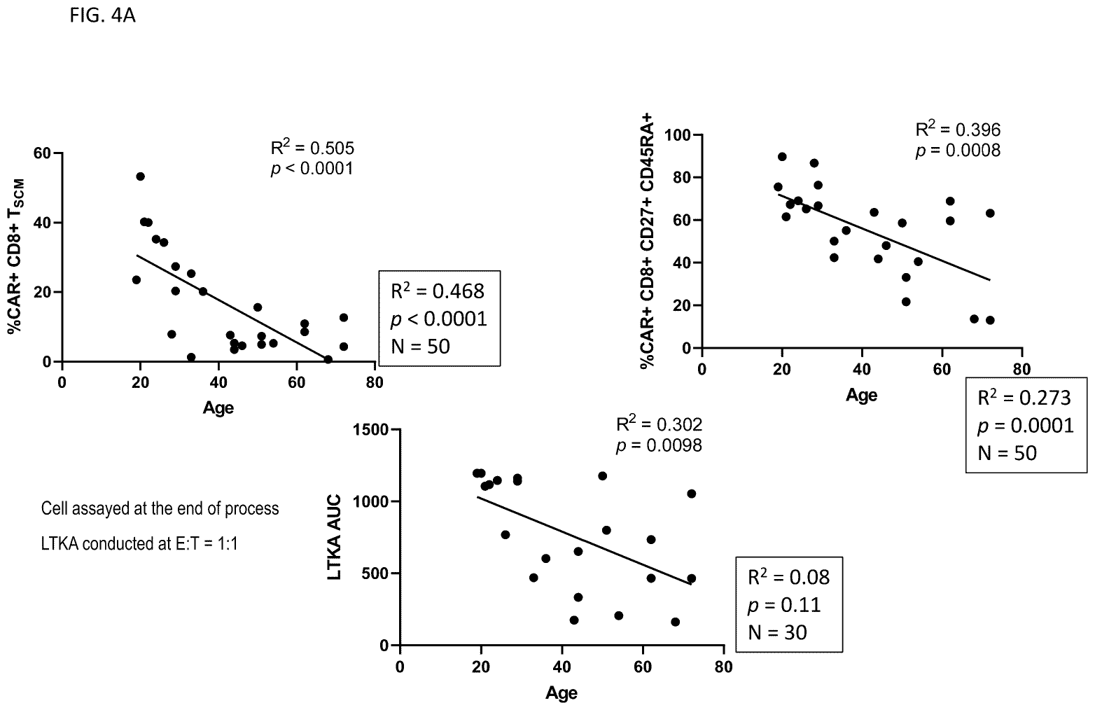

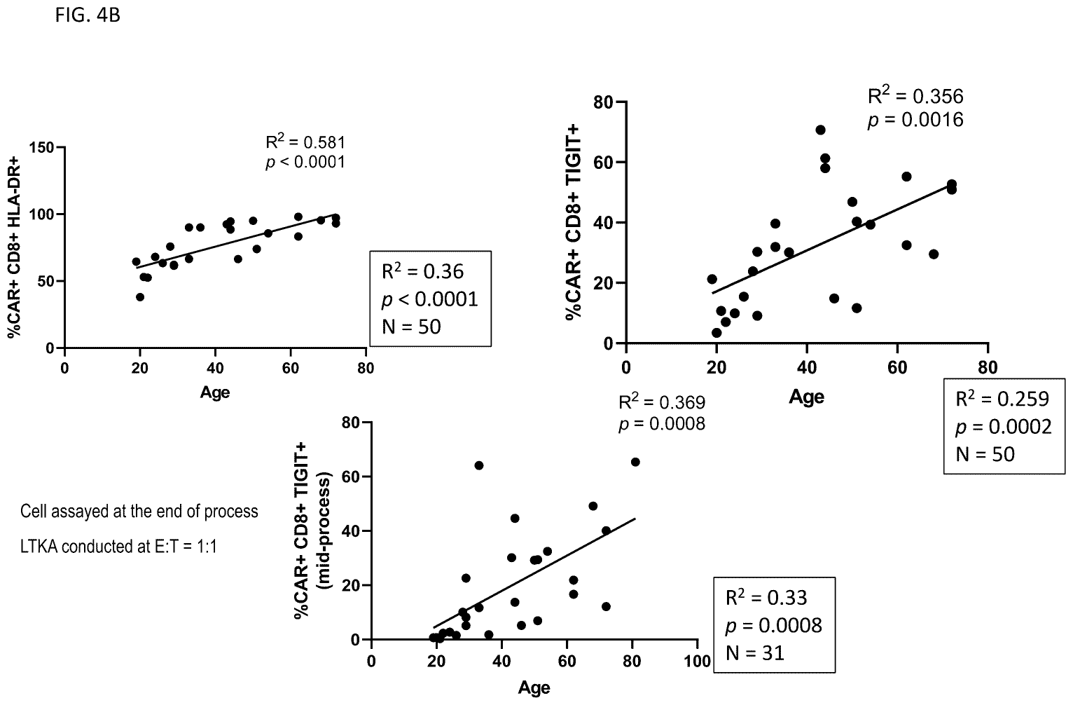

FIG. 4A-4B demonstrates that T cells from younger donors have a younger

T cell phenotype, lower expression of exhaustion marker (e.g., HLA-DR and TIGIT) and better in vitro cytotoxicity.

Statistically significant associations between expression of specific T cell activation markers and inhibitory markers, and worse in vitro anti-tumor activity, were also observed (data not shown). Expression of these specific T cell markers positively correlated with increased age. The findings in this study demonstrate the opportunity of using young healthy donor material for allogeneic CAR T products, potentially eliminating manufacturing failures and improving patient outcomes.

What is claimed is:

1. A method of manufacturing engineered immune cells comprising:
a) detecting an HLA-DR expression level of 65% or less in an immune cell population; and
b) modifying the immune cell population to express an exogenous nucleic acid sequence, thereby providing an engineered immune cell population.

2. The method of claim 1, wherein the exogenous nucleic acid sequence comprises a chimeric antigen receptor (CAR) nucleic acid sequence.

3. The method of claim 2, wherein the exogenous nucleic acid sequence further comprises one or more nucleic acid sequences selected from the group consisting of a chimeric antigen receptor (CAR), a transmembrane domain nucleic acid sequence, a costimulatory domain nucleic acid sequence and a signaling domain nucleic acid sequence.

4. The method of claim 3, wherein the exogenous nucleic acid sequence is expressed as a single transcript.

5. The method of claim 1, wherein the engineered immune cell population has improved in vitro functionality as compared to a non-engineered immune cell population.

6. The method of claim 1, wherein the engineered immune cell population has improved in vitro functionality as compared to an additional engineered immune cell population that originated from an additional immune cell population expressing HLA-DR at a level greater than about 65%.

7. The method of claim 5 or 6, wherein the improved in vitro functionality comprises one or more of improved in vitro cytotoxicity, improved cell fitness, and reduced cytokine secretion.

8. The method of claim 7, wherein the cytotoxicity is demonstrated by an in vitro killing assay.

9. The method of claim 8, wherein the cytotoxicity is demonstrated by in vitro killing assay that comprises killing of cells that express a target of the CAR.

10. The method of claim 8 or 9, wherein the in vitro killing assay is a long-term killing assay or a short-term killing assay.

11. The method of claim 8 or 9, wherein the in vitro killing assay is a long-term killing assay or a short-term killing assay.

12. The method of claim 2 or 3, wherein the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

13. The method of claim 1, wherein the modifying further comprises reducing or eliminating expression or activity of an endogenous gene.

14. The method of claim 1, wherein the immune cell population is obtained from or derived from a donor prior to the detecting step.

15. The method of claim 14, wherein the donor is a healthy donor or a patient in need of treatment.

16. The method of claim 15, wherein the patient is a patient in need of treatment with an autologous cell therapy.

17. The method of claim 16, wherein the autologous cell therapy comprises the engineered immune cell population.

18. The method of claim 1, wherein the detecting comprises detecting a protein level of HLA-DR using flow cytometry (FACS), an Enzyme-Linked Immunosorbent Assay (ELISA), an immunoblotting assay, an immunofluorescence assay, or an immunochemistry (IHC) assay.

19. The method of any one of claims 1 to 15 and 18, wherein the immune cell population is obtained from a healthy human donor.

20. The method of claim 19, wherein the healthy human donor is aged between about 18 and about 30 years old.

21. The method of any one of claims 1-20, further comprising detecting a level of expression of one or more biomarkers selected from the group consisting of TIGIT, CD16, CD56, CCR7, CD27, and CD45RA.

22. The method of any one of claims 1-20, further comprising detecting a level of expression of TIGIT.

23. The method of claim 22, wherein the TIGIT expression level is 30% or less in the immune cell population.

24. The method of any one of claims 1-23, further comprising depleting HLA-DR-positive immune cells from the immune cell population to provide an HLA-DR-depleted immune cell population.

25. The method of any one of claims 1-24, further comprising depleting TIGIT-positive immune cells from the immune cell population to provide a TIGIT-depleted immune cell population.

26. The method of claim 24 or 25, wherein the depleting step is performed prior to the modifying step.

27. An engineered immune cell population comprising 65% or less HLA-DR+ cells.

28. The engineered immune cell population of claim 27, which comprises 30% or less TIGIT+ cells.

29. The engineered immune cell population of claim 27 or 28, which comprises an exogenous nucleic acid sequence.

30. The engineered immune cell population of claim 29, which comprises an exogenous nucleic acid sequence comprising a chimeric antigen receptor (CAR) nucleic acid sequence.

31. The engineered immune cell population of claim 29, wherein the exogenous nucleic acid sequence comprises one or more nucleic acid sequences selected from the group consisting of a CAR, a transmembrane domain nucleic acid sequence, a costimulatory domain nucleic acid sequence and a signaling domain nucleic acid sequence.

32. The engineered immune cell population of any one of claims 29-31, wherein the exogenous nucleic acid sequence is expressed as a single transcript.

33. A method of manufacturing immune cells with improved in vitro functionality comprising:
a) detecting a level of HLA-DR expression in an immune cell population to provide a detected level of HLA-DR expression; and
b) modifying the immune cell population to express an exogenous nucleic acid sequence, thereby providing an engineered immune cell population,
wherein the engineered immune cell population comprises or exhibits improved in vitro functionality as compared to an additional engineered immune cell population originated from an additional immune cell population having an additional level of HLA-DR expression that is higher than the detected level.

34. The method of claim 33, wherein the detected level indicates HLA-DR is expressed in less than 65% of immune cells of the immune cell population and optionally wherein (a) the additional level of HLA-DR is expressed in more than 65% of immune cells of the additional immune cell population and/or (b) the detecting further comprises detecting a level of TIGIT expression in the immune cell population, wherein the detected TIGIT expression level is 30% or less in the immune cell population.

35. The method of claim 33, wherein the exogenous nucleic acid sequence comprises a chimeric antigen receptor (CAR) nucleic acid sequence.

36. The method of claim 35, wherein the exogenous nucleic acid sequence further comprises one or more nucleic acid sequences selected from the group consisting of a chimeric antigen receptor (CAR), a transmembrane domain nucleic acid sequence, a costimulatory domain nucleic acid sequence and a signaling domain nucleic acid sequence.

37. The method of claim 36, wherein the exogenous nucleic acid sequence is expressed as a single transcript.

38. The method of claim 33, wherein the improved in vitro functionality comprises or exhibits one or more of improved in vitro cytotoxicity, improved cell fitness, and reduced cytokine secretion.

39. The method of claim 35 or 36, wherein the improved in vitro functionality comprises or exhibits one or more of improved in vitro cytotoxicity, improved cell fitness, and reduced cytokine secretion.

40. The method of claim 38 or 39, wherein the cytotoxicity is demonstrated by an in vitro killing assay.

41. The method of claim 39, wherein the cytotoxicity is demonstrated by in vitro killing assay that comprises killing of cells that express a target of the CAR.

42. The method of claim 40, wherein the in vitro killing assay is a long-term killing assay or a short-term killing assay.

43. The method of claim 41, wherein the in vitro killing assay is a long-term killing assay or a short-term killing assay.

44. The method of claim 33, wherein the detected level indicates HLA-DR is expressed in less than 65% of immune cells of the immune cell population and wherein the additional level of HLA-DR is expressed in more than 65% of immune cells of the additional immune cell population.

45. The method of claim 35, wherein the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

46. The method of claim 33, wherein the modifying further comprises reducing or eliminating expression or activity of an endogenous gene.

47. The method of claim 33, wherein the immune cell population is obtained from or derived from a donor prior to the detecting step.

48. The method of claim 47, wherein the donor is a healthy donor or a patient in need of treatment.

49. The method of claim 48, wherein the patient is a patient in need of treatment with an autologous cell therapy.

50. The method of claim 49, wherein the autologous cell therapy comprises the engineered immune cell population.

51. The method of claim 33, wherein the detecting comprises detecting a protein level of HLA-DR using flow cytometry (FACS), an Enzyme-Linked Immunosorbent Assay (ELISA), an immunoblotting assay, an immunofluorescence assay, or an immunochemistry (IHC) assay.

52. A method of selecting a donor immune cell population for engineering comprising:
a) detecting a first level of HLA-DR expression in a first immune cell population to provide a first detected level of HLA-DR;
b) detecting a second level of HLA-DR expression in a second immune cell population to provide a second detected level of HLA-DR, wherein the second detected level is greater than the first detected level; and
c) selecting the first immune cell population for engineering.

53. The method of claim 52, wherein the first detected level indicates that HLA-DR is expressed in less than 65% of immune cells of the immune cell population.

54. The method of claim 53, wherein the second detected level indicates that HLA-DR is expressed in more than 65% of immune cells of the immune cell population.

55. The method of claim 52, further comprising modifying the first immune cell population to express an exogenous nucleic acid sequence, thereby providing an engineered immune cell population.

56. The method of claim 55, wherein the engineered immune cell population comprises or exhibits improved in vitro functionality as compared to an additional engineered immune cell population that originated from the second immune cell population.

57. The method of claim 55, wherein the exogenous nucleic acid sequence comprises a chimeric antigen receptor (CAR) nucleic acid sequence.

58. The method of claim 57, wherein the exogenous nucleic acid sequence further comprises one or more nucleic acid sequences selected from the group consisting of a CAR, a transmembrane domain nucleic acid sequence, a costimulatory domain nucleic acid sequence and a signaling domain nucleic acid sequence.

59. The method of claim 58, wherein the exogenous nucleic acid sequence is expressed as a single transcript.

60. The method of claim 56, wherein the improved in vitro functionality comprises one or more of improved in vitro cytotoxicity, improved cell fitness, and reduced cytokine secretion.

61. The method of claim 60, wherein the cytotoxicity is demonstrated by an in vitro killing assay.

62. The method of claim 60, wherein the cytotoxicity is demonstrated by in vitro killing assay that comprises killing of cells that express a target of the CAR.

63. The method of claim 61, wherein the in vitro killing assay is a long-term killing assay or a short-term killing assay.

64. The method of claim 62, wherein the in vitro killing assay is a long-term killing assay or a short-term killing assay.

65. The method of claim 52, further comprising discarding the second cell population and/or preserving the first cell population.

66. The method of claim 52, wherein the first immune cell population and/or the second immune cell population is obtained from or derived from a donor prior to the detecting step.

67. The method of claim 66, wherein the donor is a healthy donor or a patient in need of treatment.

68. The method of claim 67, wherein the patient is a patient in need of treatment with an autologous cell therapy.

69. The method of claim 68, wherein the autologous cell therapy comprises the engineered immune cell population.

70. The method of claim 52, wherein the detecting comprises detecting a protein level of HLA-DR using flow cytometry (FACS), an Enzyme-Linked Immunosorbent Assay (ELISA), an immunoblotting assay, an immunofluorescence assay, or an immunochemistry (IHC) assay.

71. The method of claim 52, wherein detecting step (a) further comprises detecting a first level of TIGIT expression in the first immune cell population to provide a first detected level of TIGIT and detecting step (b) further comprises detecting a second level of TIGIT expression in the second immune cell population to provide a second detected level of TIGIT, wherein the second detected level of TIGIT is greater than the first detected level of TIGIT.

72. The method of claim 71, wherein the first detected level of TIGIT indicates that TIGIT is expressed in less than 30% of immune cells of the immune cell population.

73. The method of claim 72, wherein the second detected level of TIGIT indicates that TIGIT is expressed in more than 30% of immune cells of the immune cell population.

74. A method of manufacturing immune cells with improved in vitro functionality comprising:
a) modifying an immune cell population to express an exogenous nucleic acid sequence, thereby providing an engineered immune cell population; and
b) depleting HLA-DR-positive engineered immune cells from the engineered immune cell population to provide an HLA-DR-depleted engineered immune cell population,
wherein the HLA-DR-depleted engineered immune cell population comprises or exhibits improved in vitro functionality as compared to an engineered immune cell population that has not been depleted of HLA-DR-positive engineered immune cells.

75. The method of claim 74, further comprising depleting additional immune cells from the engineered immune cell population, wherein the additional immune cells express one or more of TIGIT, CD16, and CD56.

76. The method of claim 74, wherein the exogenous nucleic acid sequence comprises a chimeric antigen receptor (CAR) nucleic acid sequence.

77. The method of claim 76, wherein the exogenous nucleic acid sequence further comprises one or more nucleic acid sequences selected from the group consisting of a CAR, a transmembrane domain nucleic acid sequence, a costimulatory domain nucleic acid sequence and a signaling domain nucleic acid sequence.

78. The method of claim 77, wherein the exogenous nucleic acid sequence is expressed as a single transcript.

79. The method of claim 74, wherein the improved in vitro functionality comprises one or more of improved in vitro cytotoxicity, improved cell fitness, and reduced cytokine secretion.

80. The method of claim 76 or 77, wherein the improved in vitro functionality comprises one or more of improved in vitro cytotoxicity, improved cell fitness, and reduced cytokine secretion.

81. The method of claim 79 or 80, wherein the cytotoxicity is demonstrated by an in vitro killing assay.

82. The method of claim 80, wherein the cytotoxicity is demonstrated by in vitro killing assay that comprises killing of cells that express a target of the CAR.

83. The method of claim 81, wherein the in vitro killing assay is a long-term killing assay or a short-term killing assay.

84. The method of claim 82, wherein the in vitro killing assay is a long-term killing assay or a short-term killing assay.

85. The method of claim 75, wherein the HLA-DR-depleted and TIGIT−, CD16−, or CD56-depleted engineered immune cell population comprises or exhibits improved in vitro functionality as compared to an engineered immune cell population that has not been depleted of HLA-DR-positive and TIGIT−, CD16- or CD56-positive immune cells.

86. The method of claim 76, wherein the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

87. The method of claim 74, wherein the modifying further comprises reducing or eliminating expression or activity of an endogenous gene.

88. The method of claim 74, wherein the immune cell population is obtained from or derived from a donor prior to the modifying step.

89. The method of claim 88, wherein the donor is a healthy donor or a patient in need of treatment.

90. The method of claim 89, wherein the patient is a patient in need of treatment with an autologous cell therapy.

91. The method of claim 90, wherein the autologous cell therapy comprises the engineered immune cell population.

92. The method of claim 74, wherein the depleting comprises a flow cytometry (FACS) method.

93. The method of claim 74, further comprising detecting a level of HLA-DR expression in the HLA-DR-depleted engineered immune cell population.

94. The method of claim 75, further comprising detecting a level of TIGIT expression in the TIGIT-depleted engineered immune cell population.

95. A chimeric antigen receptor T (CAR-T) cell population, in which HLA-DR is expressed at a first level and the CAR-T cell population has improved in vitro functionality as compared to a CAR-T cell population in which HLA-DR is expressed at a second level, wherein the first level is lower than the second level.

96. The CAR-T cell population of claim 95, wherein the first level is 65% or less in the CAR-T cell population and/or wherein the second level is more than 65% in the CAR-T cell population.

97. The CAR-T cell population of claim 95, wherein TIGIT is expressed at a first level and the CAR-T cell population has improved in vitro functionality as compared to a CAR-T cell population in which TIGIT is expressed at a second level, wherein the first level of TIGIT expression is lower than the second level of TIGIT expression.

98. The CAR-T cell population of claim 97, wherein the first level of TIGIT expression is 30% or less in the CAR-T cell population and/or wherein the second level of TIGIT expression is more than 30% in the CAR-T cell population.

99. The CAR-T cell population of claim 95, having an exogenous nucleic acid sequence comprising a chimeric antigen receptor (CAR) nucleic acid sequence.

100. The CAR-T cell population of claim 99, wherein the exogenous nucleic acid sequence further comprises one or more nucleic acid sequences selected from the group consisting of a CAR, a transmembrane domain nucleic acid sequence, a costimulatory domain nucleic acid sequence and a signaling domain nucleic acid sequence.

101. The CAR-T cell population of claim 100, wherein the exogenous nucleic acid sequence is expressed as a single transcript.

102. The CAR-T cell population of any one of claims 95-100, wherein the improved in vitro functionality comprises one or more of improved in vitro cytotoxicity, improved cell fitness, and reduced cytokine secretion.

103. The CAR-T cell population of claim 102, wherein the cytotoxicity is demonstrated by an in vitro killing assay.

104. The CAR-T cell population of claim 102, wherein the cytotoxicity is demonstrated by in vitro killing assay that comprises killing of cells that express a target of the CAR.

105. The CAR-T cell population of claim 103 or 104, wherein the in vitro killing assay is a long-term killing assay or a short-term killing assay.

106. The CAR-T cell population of claim 99, wherein the CAR nucleic acid sequence expresses a CAR that binds to BCMA, EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, FLT3, CD70, DLL3, CD52 or CD34.

107. A kit for in vitro functionality analysis of cell populations comprising:
a) anti-HLA-DR binding agent; and
b) instructions to use the binding agent to detect a level of HLA-DR expression in a cell population.

108. The kit of claim 107, further comprising one or more additional binding agents to detect one or more of TIGIT, CD16, and CD56.

109. The kit of claim 107, further comprising reagents for measuring in vitro cytotoxicity of a CAR T cell engineered from the cell population.

110. The kit of claim 107, wherein the binding agent is an antigen binding molecule.

111. The kit of claim 108, wherein the one or more additional binding agents are antigen binding molecules.

112. The kit of claim 109, wherein the antigen binding molecule is an antibody or fragment thereof.

113. The kit of claim 111, wherein the one or more antigen binding molecules are one or more antibodies or fragments thereof.

114. The method of any one of claims 33-51, further comprising detecting a level of expression of one or more biomarkers selected from the group consisting of TIGIT, CD16, CD56, CCR7, CD27, and CD45RA.

115. The method of any one of claims 52-70, further comprising detecting an additional first level of expression of one or more biomarkers selected from the group consisting of TIGIT, CD16, CD56, CCR7, CD27, and CD45RA.

116. The method of any one of claims 52-70 and 115, further comprising detecting an additional second level of expression of one or more biomarkers selected from the group consisting of TIGIT, CD16, CD56, CCR7, CD27, and CD45RA.

117. The method of any one of claims 74-93, further comprising depleting biomarker-positive engineered immune cells from the engineered immune cell population to provide a biomarker-depleted engineered immune cell population, wherein the biomarker is selected from the group consisting of TIGIT, CD16, and CD56.

118. The CAR-T cell population of any one of claims 95-96 and 99-106, in which a first biomarker is expressed at a third level and the CAR-T cell population has improved in vitro functionality as compared to a CAR-T cell population in which the biomarker is expressed at an fourth level, wherein the third is lower than the fourth level, wherein the first biomarker is selected from the group consisting of TIGIT, CD16, and CD56.

119. The CAR-T cell population of any one of claims 95-96, 99-106 and 118, in which an second biomarker is expressed at a fifth level and the CAR-T cell population has improved in vitro functionality as compared to a CAR-T cell population in which the biomarker is expressed at a sixth second level, wherein the fifth level is higher than the sixth level, wherein the biomarker is selected from the group consisting of CCR7, CD27, and CD45RA.

120. The method of any one of claims 33-51 and 114, wherein the immune cell population is obtained from a healthy human donor.

121. The method of any one of claims 52-73 and 115-116, wherein the first immune cell population is obtained from a healthy human donor.

122. The method of any one of claims 74-94 and 117, wherein the immune cell population is obtained from a healthy human donor.

123. The method of any one of claims 120-122, wherein the healthy human donor is aged between about 18 and about 30 years old.

124. The CAR-T cell population of any one of claims 95-106 and 118-119, wherein the CAR-T cell population was derived from a donor cell population obtained from a healthy human donor.

125. The method of claim 124, wherein the healthy human donor is aged between about 18 and about 30 years old.
